SEQUENCE LISTING (34 sequences)

SEQ ID NO: 1 - DNA, 29 bp, Artificial
  primer sequence
atgctcactacaagtaaacttggaatcag

SEQ ID NO: 2 - DNA, 30 bp, Artificial
  primer sequence
ggaggatctcattccaagtttcacttgtag

SEQ ID NO: 3 - DNA, 23 bp, Artificial
  primer sequence
aacatccctcaaaagactcaagg

SEQ ID NO: 4 - DNA, 28 bp, Artificial
  primer sequence
acatttttgctttgcaattgacaatgtc

SEQ ID NO: 5 - DNA, 26 bp, Artificial
  primer sequence
cagaagaacaactgttaacacttctc

SEQ ID NO: 6 - DNA, 29 bp, Artificial
  primer sequence
gagaagtgttaacagttgttgttcttctg

SEQ ID NO: 7 - DNA, 22 bp, Artificial
  primer sequence
aacacccctacaatggatgccg

SEQ ID NO: 8 - DNA, 25 bp, Artificial
  primer sequence
aatagtttttttcacatccaagagg

SEQ ID NO: 9 - DNA, 20 bp, Artificial
  primer sequence
ggagaagctgccaatggata

SEQ ID NO: 10 - DNA, 20 bp, Artificial
  primer sequence
ttacgcttgcacttctggtg

SEQ ID NO: 11 - DNA, 20 bp, Artificial
  primer sequence
tctgtgctttcctcgtgatg

SEQ ID NO: 12 - DNA, 20 bp, Artificial
  primer sequence
ttgagccttctggatctgct

SEQ ID NO: 13 - DNA, 20 bp, Artificial
  primer sequence
ctgcctgaaggacagaaagg

SEQ ID NO: 14 - DNA, 20 bp, Artificial
  primer sequence
tcattgagctgctgatggac

SEQ ID NO: 15 - DNA, 20 bp, Artificial
  primer sequence
cacattcacatgggtggaac

SEQ ID NO: 16 - DNA, 20 bp, Artificial
  primer sequence
tctggtgcttcctttggtct

SEQ ID NO: 17 - DNA, 20 bp, Artificial
  primer sequence
accagtgggaccactacagc

SEQ ID NO: 18 - DNA, 20 bp, Artificial
  primer sequence
atctcaagctgctggctctc

SEQ ID NO: 19 - DNA, 20 bp, Artificial
  primer sequence
tcatgggtttcatcagcaag

SEQ ID NO: 20 - DNA, 20 bp, Artificial
  primer sequence
tggtcgcatccatgatctta

SEQ ID NO: 21 - DNA, 20 bp, Artificial
  primer sequence
cttatccgaacgaccttcca

SEQ ID NO: 22 - DNA, 20 bp, Artificial
  primer sequence
cttgctgtccagatgggact

SEQ ID NO: 23 - DNA, 20 bp, Artificial
  primer sequence
cctcttgcttcaggttctgc

SEQ ID NO: 24 - DNA, 20 bp, Artificial
  primer sequence
ggcccttgtacatgatggtc

SEQ ID NO: 25 - DNA, 20 bp, Artificial
  primer sequence
ggctgtggtacccatgtttt

SEQ ID NO: 26 - DNA, 20 bp, Artificial
  primer sequence
cagaagcacggaatctgatg

SEQ ID NO: 27 - DNA, 20 bp, Artificial
  primer sequence
caaagccctggaagagtctg

SEQ ID NO: 28 - DNA, 20 bp, Artificial
  primer sequence
cggatcaggttttcagcttc

SEQ ID NO: 29 - DNA, 20 bp, Artificial
  primer sequence
aggtccctgtcatgcttctg

SEQ ID NO: 30 - DNA, 20 bp, Artificial
  primer sequence
tctggacccattccttcttg

SEQ ID NO: 31 - DNA, 20 bp, Artificial
  primer sequence
ggtctgagtgggactcaagg

SEQ ID NO: 32 - DNA, 20 bp, Artificial
  primer sequence
tctttttcatcgtggcaatg

SEQ ID NO: 33 - DNA, 20 bp, Artificial
  primer sequence
ccctcaccatcatcctcact

SEQ ID NO: 34 - DNA, 20 bp, Artificial
  primer sequence
ccttcgagtgacaaacacga

(19) United States
(12) Patent Application Publication
(10) Pub. No.: US 2010/0278864 A1
(43) Pub. Date: Nov. 4, 2010

(54) Rabies Vaccine

(75) Inventor: Zhen F. Fu, Athens, GA (US)

Correspondence Address: MUETING, RAASCH & GEBHARDT, P.A., P.O. BOX 581336, MINNEAPOLIS, MN, 55458-1336, US

(73) Assignee: University of Georgia Research Foundation, Inc., Athens, GA (US)

(21) Appl. No.: 12/086,548

(22) PCT Filed: Dec. 14, 2006
(86) PCT No.: PCT/US2006/047921

§ 371 Date: Sep. 11, 2009

Related U.S. Application Data
(60) Provisional application No. 60/750,413, filed on Dec. 14, 2005.

(51) Int. Cl. A61K 39/205; A61P 31/14; C12N 7/04

(52) U.S. Cl. 424/224.1; 435/236

(57) ABSTRACT

An attenuated rabies virus for use as a live vaccine. The attenuated rabies virus expresses an immune factor that enhances immune response upon administration of the live vaccine.

This application claims the benefit of U.S. Provisional Application Ser. No. 60/750,413, filed Dec. 14, 2005, which is incorporated herein by reference in its entirety.

### STATEMENT OF GOVERNMENT RIGHTS

This invention was made with government support under Public Health Service grant AI-051560 from the National Institute of Allergy and Infectious Diseases. The U.S. Government has certain rights in this invention.

### BACKGROUND OF THE INVENTION

Rabies virus (RV) is a highly neurotropic virus that migrates from the portal of entry to the central nervous system (CNS). It is a non-segmented negative-stranded RNA virus of the Rhabdoviridae family and induces a fatal neurological disease in humans and animals. Annually, more than 70,000 human fatalities are reported, and millions of others require post-exposure treatment. Although significant advances have been made in rabies prevention and control, the disease remains a major threat to public health and continues to cause numerous human deaths around the world. Dog remains the most important reservoir in Asia, Africa and Latin America where most human rabies cases occur. In the developed countries, human rabies has dramatically declined during the past 50 years as a direct consequence of routine vaccination of pet animals. However, wildlife rabies has emerged as a major threat. In the United States, more than 90% of animal rabies cases (>7000 per year) have been reported in wildlife, representing constant public health threats. Most of the human cases in the past decade have been associated with RV found in bats, particularly the silver-haired bats. Furthermore, most of the cases occurred without a history of exposure, suggesting that the silver-haired bat RV (SHBRV) is highly pathogenic and neuroinvasive.

All rhabdoviruses have two major structural components: a helical ribonucleoprotein core (RNP) and a surrounding envelope. The rabies genome encodes five proteins: nucleoprotein (N), phosphoprotein (P), matrix protein (M), glycoprotein (G) and polymerase (large protein) (L). The order of the genes in the wild-type rabies genome is 3′-N-P-M-G-L-5′. The N, L and P proteins are associated with the core RNP complex. The RNP complex consists of the RNA genome encapsidated by the N in combination with polymerase L and the P protein. This complex serves as a template for virus transcription and replication. The viral envelope component of RV is composed of a transmembrane glycoprotein (G) and a matrix (M) protein. The glycoprotein forms approximately 400 trimeric spikes which are tightly arranged on the surface of the virus. The M protein is associated both with the envelope and the RNP and may be the central protein of rhabdovirus assembly. The order and relative size of the genes in the rabies genome are shown in the FIG. 1. The arrangement of these proteins and the RNA genome determine the structure of the rabies virus.

RV invades the nervous system by binding to neural receptors such as acetylcholine receptor, neural cell adhesion molecule (NCAM), or nerve growth factor receptor (NTR75). Then RV is transported to the central nervous system (CNS) by retrograde transportation, possibly by binding to cytoplasmic dynein. Despite extensive investigation in the past 100 years, the pathogenic mechanisms by which street (wild-type, wt) RV infection results in neurological diseases and death in humans are not well understood. This is because there is very little neuronal pathology or damage in the CNS of rabies patients on which to base relevant mechanisms (Murphy, 1977, Arch. Virol., 54:279-297). Inflammatory reactions are mild with relatively little neuronal destruction (Miyamoto et al., 1967, J. Exp. Med., 125:447-456, Murphy 1977, Arch. Virol., 54:279-297). Laboratory attenuated RV, on the other hand, induces extensive inflammation and neuronal degeneration in experimental animals (Miyamoto et al., 1967, J. Exp. Med., 125:447-456, Yan et al., 2001, J Neurovirol 7: 518-527). However, it is not known how the attenuated and pathogenic RVs induce different host responses.

Human rabies vaccines are made from inactivated RV and have gone through successive improvements since the time of Pasteur, particularly the development of the human diploid cell culture vaccine (HDCV) (Wiktor et al., 1964. J. Immunol. 93:353-366). The tissue culture vaccine is not only safer compared to the former brain tissue vaccines by virtue of the absence of neuronal tissue, but also is more efficacious. Today, several tissue culture vaccines have been licensed with comparable efficacy and safety to HDCV, for example, the purified chicken embryo cell vaccine (PCEC) (Barth et al., 1984. J. Biol. Stand. 12:29-64; Sehgal et al., 1993. J Commun Dis. 27:36-43) and the purified Vero cell rabies vaccine (PVRV) (Suntharasamai et al., 1986. Lancet. 2:129-31). Unlike prophylactic vaccines used to protect prior to infection, RV vaccines are therapeutically administered to individuals after infection, i.e., bitten by rabid or suspected rabid animals. A typical post-exposure treatment for an individual consists of administration of multiple injections of inactivated tissue culture vaccines. In addition, it is also recommended that individuals receive equine (ERIG) or preferably humans (HRIG) anti-RV immunoglobulin (Anonymous 2000. MMWR 49:19-30). ERIG can cause serum sickness and HRIG is in short supply worldwide. Although tissue culture vaccines are safe and efficacious, they are not without problems. Because these vaccines are prepared from inactivated RV, multiple doses (at least 5) must be administered over an extended period of time (90 days) to stimulate optimal immune responses. Failure to complete the vaccine series may result in development of rabies. Allergic reactions occur in approximately 6% of the vaccinees given booster injections. Furthermore, the high cost of tissue culture vaccines makes it difficult to effectively utilize in developing countries where they are needed most. The cost of post-exposure treatment including vaccine and anti-RV immunoglobulin is estimated to be more than US$3,000 per case. Most human cases occur in developing countries where victims cannot afford such treatment. As a result, the most frequently used vaccines in developing countries are derived from animal origin usually produced from suckling mouse (Fuenzelida vaccine) or sheep brains, which may cause neurological reactions because of contamination of neuronal tissue.

Less frequently, humans considered to be at risk of apparent rabies exposure, such as animal care control officers, veterinarians, and laboratory personnel, are routinely immunized against rabies, which is referred to as pre-exposure prophylaxis. Routine vaccination of pet animals (dogs and cats) is carried out at 3 months of age and the animals are revaccinated annually or triennially depending on the vaccine used. Licensed rabies vaccines for pets are also manufactured from inactivated viruses. Recently, a recombinant canarypox virus expressing RV glycoprotein (G) was approved for cats. Although these vaccines provide adequate protection, they do induce local reactions. In addition, multiple immunizations are required to maintain sufficient immunity throughout the life of the animal. Furthermore, vaccination of puppies <3 months of age fails to induce protective immunity, although maternal antibodies declined to undetectable levels by 6 wk of age. There is a period from the time of the waning maternal antibody to the time of active immunity during which the young animals may not be protected.

Wildlife rabies exists in many countries and continues to present a major public health threat. Efforts to control wildlife rabies during the past three decades in Europe and North America have been directed towards oral vaccination. Previously, an attenuated RV, Street Alabama Dufferin (SAD) B19, was used in Europe, which resulted in immunization of foxes and stopped RV spread to untreated areas (Wandeler et al., 1998. Rev. Infect. Dis. 10:S649-S653; Schneider et al., Rev. Infect. Dis. 10:S654-S659). However, SAD can cause disease in rodents and domestic animals. Subsequently, a recombinant vaccinia virus expressing the RV G (VRG) was developed (Kieny et al., 1984. Nature. 312:163-166) and was found to be an effective oral immunogen for raccoons and foxes under laboratory setting and in the field. Application of VRG resulted in large scale elimination of fox rabies in parts of Europe. Similar applications of VRG in the United States resulted in a blockade of coyote rabies spread in Texas and raccoon rabies spread in other states. Although VRG is safe in vaccinated animals, and efficacious in stimulating active immunity, an incident involving vaccine-induced illness in a pregnant woman underscores the risks of using such recombinant vaccines. The pregnant woman was bitten when she was removing VRG-laden bait from her dog's mouth. Within 10 days she developed an intensive local inflammatory reaction and then generalized erythroderma that subsided eventually after exfoliation (Rupprecht et al., 2001. N Engl 3 Med. 345:582-6). This incidence casts doubts on the future use of VRG as a rabies vaccine.

Based on the brief review outlined above, it is clear that current RV vaccines have problems not only with cost, but also with safety. More effective, safe, and affordable vaccines are needed. Many novel vaccines are being developed and tested including DNA and other recombinant vaccines. DNA vectors expressing RV G have been found to stimulate both humoral and cell-mediated immunity (Xiang et al., 1994. Virology. 199:132-140). Furthermore, immunization with these DNA vectors protected mice and monkeys against challenge infections. However, induction of immune responses by DNA vaccines usually takes longer, and the magnitude of the immune response is lower when compared with conventional vaccines (Lodmell et al., 1998. Nat Med. 4:949-952; Osorio et al., 1999. Vaccine. 17:1109-1116). Recombinant human adenovirus expressing RV G has also been developed (Prevec et al., 1990. J. Infect. Dis. 161:27-30; Xiang et al., 1996. Virology. 219:220-227). The recombinant adenovirus vaccines induce virus neutralizing antibodies (VNA) and protect vaccinated animals (mice, dogs, skunks, and foxes) against challenge infection. However, there is concern that preexisting anti-adenoviral immunity may prevent the uptake of the vaccine, thus, impair the active immune response.

Live attenuated viral vaccines have long been known to be more effective in inducing long-lasting cell-mediated and humoral immunity, and many diseases are controlled or eradicated by using live modified virus vaccines. It is hypothesized that a live modified RV vaccine may have an advantage over currently licensed inactivated vaccines by providing longer duration of immunity with reduced vaccine doses. As a result, the cost of vaccination will be greatly lowered. However, such a modified live RV vaccine must be completely avirulent, particularly for humans. To this end the SAD strain of the RV, which was initially used for wildlife vaccination, was further attenuated by successive selection using neutralizing monoclonal antibodies (Mab), resulting in mutation of arginine 333 to glutamic acid. One of these mutants, SAG2, has been shown to be avirulent for adult rodents, foxes, cats, and dogs by intracerebral (IC) route of infection (Le Blois et al., 1990. Vet. Microbiol. 23:159-166; Schumacher et al., 1993. Onderstepoort J. Bet. Res. 60:459-462). Oral vaccination of dogs and cats has resulted in protection against lethal challenge. Field trials with SAG2 in immunizing foxes and dogs demonstrated its safety and immunogenicity. However, the immunogenicity of SAG2 is low and SAG2 can induce disease in suckling mice when inoculated by IC, raising the possibility that young or immunocompromised individuals may still be susceptible to the virus and develop disease. Thus increased immunogenicity is required for such RV to be developed as a live avirulent vaccine.

With recent development of reverse genetics technology, manipulation of the viral genome for negative-stranded RNA viruses became possible. Application of this technology has resulted in attenuation of many viruses and some of them could be developed as live attenuated vaccines. For example, rearrangement and relocation of VSV genes resulted in reduced pathogenicity and increased immunogenicity (Wertz et al., 1998. Proc. Natl. Acad. Sci. USA. 95:3501-3506; Flanagan et al., 2000. J. Viral. 74:7895-7902). In RV, gene mutation on both the P and G (Mebatsion et al., 2001. J Virol. 75:11496-502), deletion of the P (Shoji et al., 2004. Virology. 318:295-305), and addition of cytochrome C (Faber et al., 2002. J Virol. 76:3374-81) or an extra copy of RV G (Pulmanausahakul et al., 2001. J Virol. 75:10800-7) have been carried out on RV genome for development of attenuated RV vaccines. Most of these vaccines are efficacious in inducing protective immune responses. Patents have been issued to some of these modified live attenuated rabies virus vaccines including Mebatsion (U.S. Pat. No. 6,887,479, issued May 3, 2005) and Dietzschold et al. (U.S. Pat. No. 7,074,413, issued Jul. 11, 2006). However, some of these RV can still induce diseases in suckling mice as SAG2. In our laboratory (Wu et al., 2001, J. Virol. 76:4153-5161), we showed that mutation of the N gene at the phosphorylation site reduced the rate of replication by 90% and virus production by 10,000 times compared to the parental virus.

Interferons, such as IFN-α, β, and γ, are critical anti-viral components in the innate immune system. Viral products, particularly double-stranded RNA, activate NF-κB, interferon regulatory factors (IRFs), and AP-1, leading to the production of interferons (IFNs), particularly IFN-β. IFNs, by binding to IFN receptors, activate signal transducer and activation of transcription (STAT) family of proteins, leading to induction of antiviral state. IFNs activate RNA-dependent protein kinase (PKR) which in turn phosphorylates eukaryotic translation initiation factor (eIF-2α), resulting in inhibition of mRNA translation. IFNs activate 2′5′-oligoadenylate synthetase (OAS) which in turn activates RNase L, leading to RNA degradation. IFNs up-regulate the expression of protein GTPase Mx proteins, exerting anti-viral functions. IFNs also induce the expression of inducible nitric oxide synthase (iNOS) and MHC class I and II molecules. Activated STAT can also induce the expression of chemokines which can have direct anti-viral activities and/or recruit inflammatory cells to the site of infection to kill virus-infected cells.

Chemokines are a family of structurally related proteins that mediate leukocyte activation and/or chemotactic activity. The majority of chemokines have molecular masses of 8-10 kDa and show approximately 20-50% sequence homology among each other at the protein level. Chemokine proteins also share common gene sequences and tertiary structures, and all chemokines possess a number of conserved cysteine residues involved in intramolecular disulfide bond formation. Chemokines can be divided into four major subfamilies based on cysteine signature motifs: the C, CC, CXC and CX3C families. Chemokines in which the C1 and C2 cysteine residues are separated by a single amino acid are called CXC chemokines, and include IL-8, IP-10, I-TAC and SDF-1. CXC chemokines act as chemoattractants for neutrophils and have been shown to be important mediators of T- and B-lymphocyte chemotaxis. Chemokines in which the C1 and C2 cysteine residues are adjacent are called CC chemokines, and include RANTES, MCP-1, TARC and eotaxin. Many CC chemokines exert their effects on monocytes and macrophages, but CC chemokines have been shown to be important for dendritic cell chemotaxis and some CC chemokines appear to act preferentially on Th2-type T cells. Chemokines mediate their effects by binding to G protein coupled receptors. Upon binding, the chemokine receptors initiate cellular signaling through changes in intracellular concentrations of calcium and cAMP. Many cellular chemokine receptors can bind more than one chemokine with similar affinities. For example, the chemokine receptors CCR1 and CCRS may bind RANTES, MIP-1α and MIP-1β, whereas the chemokine receptors CXCR1 and CXCR2 may bind IL-8. Viral infection of the CNS can result in a temporal expression of several chemokines and chemokine receptors by resident cells of the CNS as well as by inflammatory cells. Viral infection of glial cells results in robust expression of numerous CC chemokines such as CCL2, CCL3, CCL4, and CCL5 following infection with measles virus (Xiao et al., 1998. J Neurocytol 27(8):575-580), mouse hepatitis virus (Lane et al., 1998. J Immunol 1998;160:970-978), and human coronavirus (Edwards et al., 2000. J Neuroimmunol. 108(1-2):73-81). Infection of rat astrocytes and microglia with paramyxoviruses resulted in rapid expression of mRNA transcripts for CCL5 and CXCL10 (Vanguri et al., 1994. J Immunol 152(3):1411-1418; Fisher et al., 1995. Brain Behav Immun. 9(4):331-344). CXCL10 has also been reported to be activated by macrophages infected with RV (Nakamichi et al., 2004. J. Virol. 78:9376-88). Induction of chemokine and/or chemokine receptor expression has been reported to be beneficial to the host by attracting T lymphocytes and macrophages which participate in host defense through elimination of the invading virus (Alcami. 2003. Nat Rev Immunol. 3:36-50; Chensue, 2001. Clin Microbiol Rev. 14:821-35).

Incorporation of cytokine or chemokine genes into vaccine candidates has been investigated. For example, granulocyte-macrophage colony stimulation factor (GM-CSF) has been utilized as a vaccine immune adjuvant because of its ability to stimulate Langerhans' and dendritic cells (DCs) (Witmer-Pack et al., 1987. J Exp Med 166:1484-1498; Romani et al., 1989. J Exp Med 169:1169-1178; Ahmed et al., 2005. Mol Immunol. 42:251-8). IFN and other chemokine genes have also been cloned into viruses that resulted in reduction of virulence and enhanced immune responses (Legrand et al., 2005. Proc Natl Acad Sci U S A. 102:2940-5; Ahlers et al., 2003. Curr Mol Med. 3:285-301). Furthermore, plasmids expressing IFN or chemokines have been found to have direct anti-viral activities as well as improve the immunogenicity of other DNA or vector vaccines (Barouch et al., 2003. J Virol. 77:8729-35; Bartlett et al., 2003. 5:43-52; Biragyn et al., 2002. Blood. 100:1153-9).

Rabies remains a major public health threat around the world. Controlling rabies and protecting humans from rabies requires multi-layered control strategies, such as routine immunization of pet animals and wildlife carriers, pre-exposure immunization of people at risk, and post-exposure treatment of people bitten by rabid animals. Although inactivated rabies virus (RV) vaccines prepared from cell culture are safe and efficacious, they have disadvantages. These vaccines are expensive and thus beyond the reach of most people who need the vaccines in the developing countries. Furthermore, repeated immunizations with inactivated vaccines are required to produce a protective immune response. In addition, these inactivated vaccines always include adjuvants which may cause side effects. Thus, safer, cheaper, and more efficacious RV vaccines are needed.

### SUMMARY OF THE INVENTION

The invention provides a live attenuated rabies virus for use in a vaccine for the treatment and prevention of rabies. The attenuated rabies virus of the invention produces an inflammatory response in a mammalian subject and activates innate immune and/or antiviral responses. It may induce immune responses in the central nervous system (CNS) and help clear the virus from the CNS. The attenuated rabies virus may induce the expression of host genes involved in the innate immune and/or antiviral responses, particularly those related to alpha/beta interferon (IFN-α/β) signaling pathways and inflammatory chemokines. Many of the interferon regulatory genes, such as the signal transduction activation transducers and interferon regulatory factors, as well as the effector genes, for example 2′-5′-oligoadenylate synthetase and myxovirus proteins, may be highly induced by the vaccine in a mammalian subject.

In a preferred embodiment, the attenuated rabies virus is a recombinant virus that has been genetically modified to express one or more immune factors such as cytokines, chemokines and/or interferons so as to enhance the immunogenicity of the attenuated virus. Exemplary immune factors are described herein at many locations throughout the application, including the Background section and the Examples, and it should be understood that any of them can be expressed by the attenuated rabies virus of the invention. Chemokines can, for example, include any of the C, CC, CXC and CX3C type chemokines, as described above. Examples of preferred transgenes that can be introduced into the attenuated rabies virus include, without limitation, polynucleotides operably encoding interferons such as IFN-α2, IFN-α4, IFN-α5, IFN-β and IFN-yγ and chemokines such as MIP-1α, MIP-1β, MCP, RANTES and IP-10. In some embodiments, the attenuated rabies virus also expresses a Toll-like receptor (TLR) and/or a TLR adaptor molecule such as TRIF or Myd88. Avirulent vaccines are prepared by cloning and expressing the immune factor(s) in RV constructs. By combining attenuation with expression of one or more immune factors, an avirulent RV vaccine can be produced that stimulates both the innate and enhances adaptive immune responses, such as the production of neutralizing antibodies. We have shown that attenuated RV induces host innate immune responses (e.g., chemokines and IFN) in the spinal cord, which blocked virus from spreading to the brain (e.g., Examples II, IV). Therefore, recombinant RV expressing IFN or chemokines should not only be able to prevent RV spread but also enhance the adaptive immune responses that are highly desired for use of RV vaccination in post-exposure treatment.

The vaccine of the invention can be administered prophylactically, prior to exposure to the rabies virus, or it can be administered as a therapeutic after exposure to the virus. If administered after exposure to the rabies virus, it is preferably administered as soon after exposure as possible, prior to the appearance of symptoms. The vaccine may also be administered after appearance of symptoms.

Accordingly, the invention provides an attenuated rabies virus that includes polynucleotide operably encoding at least one mammalian immune factor. Optionally, the polynucleotide operably encodes a plurality of mammalian immune factors. A preferred immune factor includes an interferon (such as IFN-α, IFN-β, or IFN-γ), a cytokine, a chemokine (such as MIP-1α, MIP-1β, MCP, RANTES or IP-10), a Toll-like receptor (TLR), or TLR adaptor molecule.

The invention further provides a pharmaceutical composition that includes the attenuated rabies virus and a pharmaceutically acceptable carrier, as well as a method for vaccinating a mammalian subject that involves administering to the mammalian subject the pharmaceutical composition of the invention. The pharmaceutical composition is useful as a live vaccine for the vaccination of a mammalian subject against rabies. The subject is preferably human but may be a domestic or wild animal. The pharmaceutical composition can be administered either before or after exposure to a pathogenic rabies virus. Preferably, following administration to a mammalian host, the pharmaceutical composition induces an immune response in the host that blocks or inhibits the spread of a pathogenic rabies virus within the central nervous system of the host. The amount of the attenuated rabies virus included in the pharmaceutical composition is preferably effective to induce neutralizing antibodies in a subject and/or protect the subject against experimental or natural challenge with a pathogenic rabies virus.

Unless otherwise specified, “a,” “an,” “the,” and “at least one” are used interchangeably and mean one or more than one.

### BRIEF DESCRIPTION OF THE DRAWINGS

FIG. 1 shows the rabies virus genome.

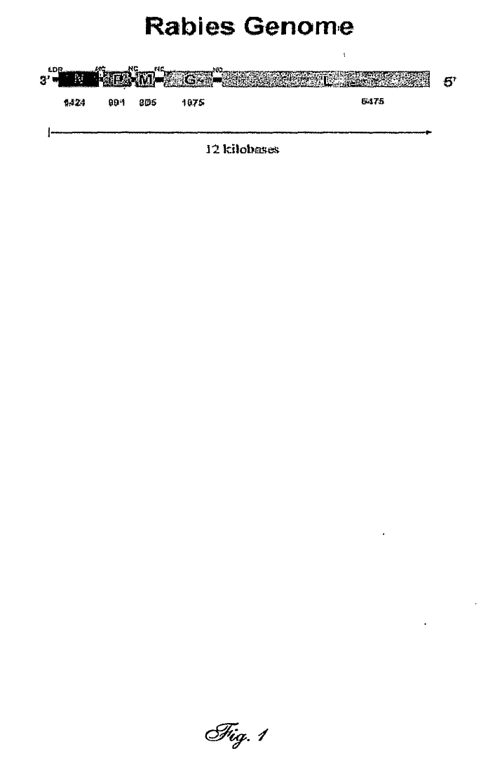

FIG. 2 shows that SHBRV is more pathogenic and induces less inflammation than B2C. The pathogenic indices for B2C and SHBRV were determined by subtracting the log virus titer/ml in BHK cells from the log ICLD₅₀/ml or the log IMLD₅₀/ml (A). Pathological changes in the cortex (IC) or the thalamus (IM) were observed in paraffin-sections infected with either B2C or SHBRV (B). Hippocampal sections were made for immunohistochemistry to quantify CD-3 positive cells using anit-CD3 antibodies (C). Con; sham-infected control mouse brain, B2C, B2C-infected mouse brain, SHB: SHBRV-infected mouse brain, (*P<0.05 SHB vs. Con, ** P<0.01 B2C vs. SHB and p<0.001 B2C vs. Con).

FIG. 3 shows expression pattern of host genes. Hierarchical cluster analysis of host gene expression was performed by gene ontology. The results were filtered to retain only those genes involved in the immunity and antiviral, apoptosis, neuron-specific, and transcriptional factors. Open bars: up-regulation, shaded bars: down-regulation.

FIG. 4 shows expression of STAT proteins in mouse brain or in primary neuronal cell culture after RV infection. STAT (1, 2, and 3) proteins were detected by Western blotting in brain tissues from mice infected by the IC or IM routes as well as in primary neurons. As a loading control, β-tubulin was detected in the same sample preparation using anti-β-tubulin (anti-T) antibody in the Western blots. The protein band intensity was determined by densitometry using that from the control as 100%.

FIG. 5 shows nuclear translocation of STAT proteins in primary neurons after RV infection. Primary neurons were infected with each of the viruses at 0.1 ffu/cell and then fixed at day 5 after infection. Viral antigen (N) was detected by using FITC-conjugated anti-RV N monoclonal antibodies. The expression of STAT1, STAT2, and STATS was detected by using rabbit anti-STAT polyclonal Abs and anti-rabbit secondary antibody conjugated with Alexa 488. Propidium Iodide (PI) was used for counter staining. The cells were examined under a Leica TCS NT confocal microscope (A). The percentage of cells with nuclear translocation was quantified for each of the STAT proteins (B). Significantly more nuclear translocated cells were observed for STAT1 (p<0.001) and STAT2 (p<0.001) in cells infected with B2C, and for STAT2 (p<0.01) in cells infected with SHBRV (as indicated by **).

FIG. 6 shows expression of RV proteins in mouse brain or in primary neuronal culture after RV infection. RV proteins (N and G) were detected by immunohistochemistry in mouse brain sections (A) or by Western blotting in brain tissues from mice infected by the IC or IM routes as well as in primary neurons (B). As a loading control, tubulin was detected in the same sample preparation using anti-β-tubulin (anti-T) antibody in the Western blots. The ratio between G and N was determined after measurement of the band density.

FIG. 7 shows RV sensitivity to IFN treatment. NA cells were treated with FN-α 24 hours before the cells with infected with 1 ffu of B2C or SHBRV. After 48 hours incubation, the supernatants were harvested for virus titration in NA cells.

FIG. 8 shows attenuated RV induces more inflammation than virulent RV. Cortical sections were made for immunohistochemistry to quantify CD-3 positive cells using anit-CD3 antibodies. (*P<0.05 SHB vs. Con, ** P<0.01 L/16/B2C vs. SHB and P<0.001 L16/B2C vs. Con).

FIG. 9 shows detection of apoptosis, pathological changes, and viral antigens in mice infected with different RVs. Mice were infected with 10 ICLD50 of each virus and brains were harvested for histopathology (HISTO) and detection of apoptosis using TUNEL assay (TUNEL, arrows indicate TUNEL-positive cells). Viral antigens (RV-N and RV-G) were detected using anti-G and anti-N antibodies as described in the text. (magnification 40×).

FIG. 10 shows inverse correlation between the induction of apoptosis and pathogenicity. Pathogenic index were plotted against the numbers of apoptotic cells observed in mice infected with different RVs using SigmaPlot. Vertical bars indicate the standard deviation.

FIG. 11 shows induction of apoptosis by RVs in primary neurons. Primary neurons were infected with each of the viruses and apoptosis was detected using the TUNEL assay. The number of apoptotic neurons was assayed and analyzed using one way ANOVA at the p<0.05 level.

FIG. 12 shows survival rate in mice infected with different RVs at different doses. Mice (10 in each group) were infected with different doses of SHBRV or B2C and the development of rabies were recorded daily for 20 days. No animals died after 12 days p.i.

FIG. 13 shows induction of apoptosis in the spinal cords. Spinal cord tissues from mice infected with B2C (A) or SHBRV (B) by the IM route at day 7 p.i. were fixed for detection of apoptosis using TUNEL assay (Magnification 40×). Spinal cord from sham-infected mice was included as control (C). Double labeling was performed to detected viral antigen and apoptosis in the spinal cord of mice infected with B2C at day 7 p.i. (D). Both viral antigen and TUNEL-positive neurons are shown by arrows (Magnification 100×). Ventral horn of a spinal cord from B2C-infected mouse stained with cresyl violet showing condensations of nuclear chromatin and neurophagia (arrows) (E, Magnification 100×).

FIG. 14 shows construction of recombinant rabies virus expressing mouse MIP-1α (HEP-MIP1α). Note that the MIP-1α is cloned between the G and L genes of the parental rabies virus (rHEP).

FIG. 15 shows growth curves of parental virus (rHEP) and recombinant viruses (HEP-MIP1 alpha, HEP-RANTES, HEP-IP10, and HEP-IFN beta).

FIG. 16 shows detection of MIP-1α in mock-infected cells or cells infected with either parental virus (rHEP) or recombinant virus expressing MIP-1 α (HEP-MIP1 alpha) at different doses. MIP-1α is only detected in cells infected with recombinant virus.

FIG. 17 shows recombinant RVs designed to express IFN or chemokines.

FIG. 18 shows recombinant RVs designed with N mutation (V), both N and G mutations (VI), and G relocation (VII).

### DETAILED DESCRIPTION OF THE INVENTION

The invention provides a pharmaceutical composition for use as a live vaccine for treatment and/or prevention of rabies, as well as methods for making and using a live vaccine. The rabies vaccine of the invention includes a live attenuated rabies virus (RV). Surprisingly, the attenuated rabies virus of the invention activates, while pathogenic rabies virus evades, innate immune and antiviral responses in the host.

In a particularly preferred embodiment, the attenuated RV is genetically engineered to contain one or more transgenes that express a mammalian immune factor. An immune factor is a molecule that stimulates or enhances the host's immune response. An immune factor expressed by the attenuated RV may be similar or identical to an immune factor naturally produced in the host organism. Examples of immune factors include cytokines, chemokines, interferons (IFNs), Toll-like receptors (TLRs), and TLR adaptor molecules (TRIF, Myd88, for example).

Attenuated RV, and recombinant attenuated RV expressing one or more immune factors, are useful as live and avirulent RV vaccines with enhanced efficacy. Avirulent RV vaccines of the invention with enhanced immunogenicity may be used prophylactically or therapeutically. These constructs are expected not only to enhance the adaptive immune responses, but also induce innate immune responses that can block the spread of virulent RV from the spinal cord to the brain, which is important in post-exposure treatment. The induced innate immune response can thus reduce the virulence of rabies virus and can enhance the host's adaptive immunity (production of neutralizing antibodies), thereby increasing the immunogenicity. Because of the host immune responses induced by an attenuated virus expressing one or more immune factors, the vaccine of the invention provides better protection than other attenuated rabies virus vaccines.

The invention is further directed to attenuated rabies viruses, as well as methods of making and using attenuated rabies viruses. Preferred attenuated rabies viruses are those that contain one or more transgenes that express a mammalian immune factor, such as a cytokine, chemokine and/or interferon.

The term “attenuated rabies virus” refers to a rabies virus that has been genetically modified in a way that reduces its pathogenicity in a warm-blooded animal, such as a mammal. Typically, an attenuated RV exhibits reduced ability to spread in the central nervous system (CNS) compared to wild-type RV. While RV attenuation is marked by reduced neuroinvasiveness, viral transcription and replication is usually not reduced.

Genetic modification of a rabies virus to produce an attenuated RV is generally accomplished using standard recombinant techniques, including reverse genetics technology, site-directed mutagenesis, gene shuffling, and the like, as exemplified in the Examples below.

The pathogenicity of rabies virus relates to its ability to invade the central nervous system and cause neurological disease and death. A reduction in the pathogenicity of a rabies virus can be observed, determined or measured in any of a number of ways, such as by detecting reductions in weight loss, morbidity such as ruffed fur and paralysis, and mortality. Typically, pathogenicity is evaluated by administering the attenuated rabies virus to a mammalian host, for example a population of adult mice or suckling mice, and observing the resulting morbidity or mortality in the mammalian host population. The pathogenicity of the mutant rabies viruses can be compared with that of the parent viruses by inoculating mice, typically intramuscularly, intracranially or intracerebally. Preferred attenuated viruses do not induce disease in adult animals, and preferably do not induce any disease in neonatal animals after intracerebral inoculation.

Attenuated rabies viruses may, for example, have mutations in one or more of the constituent viral proteins, particularly the G protein, the N protein, and/or the P protein. The arginine (R) or lysine (K) residue at position 333 (R333/K333) of the G protein has been shown to be associated with RV pathogenicity (D333 or E333 is much less pathogenic) (Dietzschold et al., 1983, Proc Natl Acad Sci U S A. 80:70-4; Coulon et al., J Gen Virol. 1983, 64:693-6). Stable attenuated RV mutants which replace the Arg at position 333 of the G protein with other amino acids are described in WO00/32755 and Morimoto et al. (2001, Vaccine 19:3543-51). Rabies viruses possessing an amino acid other than R333 or L333 in the glycoprotein are nonpathogenic to immunocompetent adult mice. However, they may still be pathogenic when inoculated into baby mice, demonstrating the existence of residual pathogenicity and the potential risk to immunocompromised animals and humans.

Mutations in the LC8 binding domain of the P protein also have been shown to reduce pathogenicity, particularly when they occur together with a mutation at R333 of the P protein. Interactions between P protein of the rabies virus and LC8 receptor protein of the host appear to be involved in retrograde transport of RV from the peripheral nervous system to the central nervous system and thus pathogenesis of RV. Mebatsion (J. Virol., 2001, 75:11496-11502) found that when deletions were introduced into the LC8 binding site of the G protein in a rabies virus that possesses a P protein having an amino acid differing from R333, a dramatic reduction in pathogenicity for 1- to 2-day-old suckling mice was observed after peripheral inoculation. The SAD-D29-derived deletion mutants described in Mebastion were found to be attenuated by as much as 30-fold after intramuscular inoculation but remained as pathogenic as the parent virus when inoculated directly into the brain, suggesting that abolishing the P-LC8 interaction reduces the efficiency of peripheral spread of the more attenuated SAD-D29 strain.

Example II shows construction of mutations in both the G and N genes which resulted in the reduction of neuroinvasiveness as well as viral replication. Further, we have found that attenuated RV expresses at least three-fold more of the G than wt RV both in vitro and in vivo, while the expression of the N protein is similar. Without intending to be bound by theory, it is believed that wt RV evades the innate immunity of the host by reducing the level of G expression; thus, attenuated RV that express high levels of G are particularly well-suited for use in the rabies vaccine of the invention.

A preferred attenuated virus contains mutations (i.e., deletions, insertions, substitutions or rearrangements) in two or more rabies proteins. Other preferred attenuated viruses are described in Schneider (U.S. Pat. No. 4,752,474, issued Jun. 21, 1988), Mebatsion (U.S. Pat. No. 6,887,479, issued May 3, 2005) and Dietzschold et al. (U.S. Pat. No. 7,074,413, issued Jul. 11, 2006).

In some attenuated rabies viruses, antigenic determinants that render the rabies virus non-pathogenic are combined with determinants that are responsible for the elicitation of an effective anti-rabies immune response. See Dietzschold et al. (U.S. Pat. No. 7,074,413, issued Jul. 11, 2006). Additionally or alternatively, an attenuated rabies virus may have a genome in which the genes encoding the rabies viral proteins are present in a different order compared to the order in which they appear in the wild-type virus. Attenuated rabies viruses may also exhibit deletions or insertions in the rabies genome, and/or may express one or more transgenes, as exemplified herein.

The attenuated rabies virus of the invention, when used as a component of a rabies vaccine, is not limited to any particular attenuated RV described herein but can include any attenuated rabies virus. Examples of attenuated RVs that can be used in the vaccine of the invention include attenuated viruses based on SAD (e.g., L16, B19, Berne, D29), SAG (1 and 2), Flury LEP (low egg passage), Flury HEP (high egg passage), CVS (e.g., AV01, 11, 27), ERA, RC-HL and AG, among others (see, e.g., Clark et al., 1972. Rabies virus, p.177-182. In M. Majer and S. A. Plotkin (ed.), Strains of human viruses. S. Karger, Basel). SAD-L16 (L16), SAD-D29 (D29) and mutants thereof are described in Mebatsion, Journal of Virology, 2001, 75:11496-11502; SAD-B19 is described in Conzelmann et al., Virol, 1990 175:485-99); and SAG-2 is described in Schumacher et al., Onderstepoort J Vet Res. 1993 December;60(4):459-62). CVS-B2C is a laboratory-adapted, attenuated virus isolated from CVS-24 virus by passaging in BHK cells (Morimoto et al., 2000, J Neurovirol. 6:373-81). Strains L16-G (essentially SAG-2; both bear the R333E mutation on the G), L16N, L16Q, L-16Q-G, HEP, and others are described in the Examples below. Particularly preferred attenuated rabies viruses are those that, while exhibiting little or no pathogenicity, are able to propagate in cell culture as efficiently as the parent strains.

In a preferred embodiment that is especially useful in the formulation of a live vaccine, the attenuated rabies virus has been genetically modified to express one or more cytokines, chemokines and/or interferons so as to enhance the immunogenicity of the attenuated virus. Examples of transgenes that can be introduced into the attenuated rabies virus include, without limitation, polynucleotides operably encoding interferons such as IFN-_60 (e.g., IFN-α2 and IFN-α5) IFN-β and IFN-γ; chemokines such as MIP-1α, 1α, MIP-1β, MCP, RANTES and IP-10; cytokines such as interleukin-12 (IL-12), granulocyte-macrophage colony-stimulating factor (GM-CSF), interleukin-6 (IL-6), interleukin-18 (IL-18) and tumor necrosis factor (TNF); Toll like receptor (TLR1-9) and adaptor molecules (TRIF, Myd88, etc). In one embodiment, the attenuated RV expresses one or more interferons, but does not express a cytokine or a chemokine. In another embodiment, the attenuated RV expresses a cytokine and/or a chemokine, but does not express an interferon. In yet another embodiment, the attenuated RV expresses at least one interferon and at least on cytokine or chemokine. The IFN and/or specific chemokines are expected to induce the innate and enhance the adaptive immune responses in RV vaccination. It is further expected that IFN and/or chemokine expression will have anti-viral function and recruit appropriate immune cells into the CNS to clear RV-infected cells. If RV gets into peripheral nervous system (PNS) of a subject that has been vaccinated with a live attenuated rabies vaccine of the invention, it is expected that the virus will be killed before it gets into the CNS. These expectations are based on our observation that attenuated RV activated innate immune responses including up-regulation of IFN and chemokine expression. In contrast, wild-type (wt) RV was found to evade the innate immune responses. See Example H, for example.

In some embodiments, the attenuated rabies virus has been further genetically modified to expresses a Toll-like receptor (TLR) or a TLR adaptor molecule such as TRIF or MyD88. TIR domain-containing adapter-inducing IFN-β (TRIF), also known as TIR-containing adaptor molecule (EICAM)-1, is an adaptor involved in the MyD88 independent signaling pathway of some TLRs. Myeloid differentiation factor 88 (MyD88) is a cytoplasmic adaptor protein that is involved in IL-1 receptor (IL-1R)- and TLR-induced activation of NF-κB. Expression of TRIF increases cellular immune response, such as T cell response, in the host, whereas MyD88 enhances the host's antigen-specific humoral response, such as antibody production. Expression of an adaptor molecule may be particularly advantageous in recombinant RV constructs that otherwise would be characterized by diminished antigen. Constructs that induce innate immunity but are characterized by reduced virus replication may result in lower antigen mass, particularly for those constructs that express IFN since IFN has direct anti-viral activities. On the other hand, for those constructs that express chemokines, antigen mass is not expected to be reduced because of the time it takes chemokines to induce inflammatory responses. Engulfment of virus-infected cells will help, not hurt, antigen presentation, thus increase the adaptive immune responses.

The vaccine of the invention produces an inflammatory response in a mammalian subject. It also induces the expression of host genes involved in the innate immune and antiviral responses, particularly those related to alpha/beta interferon (IFN-α/β) signaling pathways and inflammatory chemokines. Many of the interferon regulatory genes, such as the signal transduction activation transducers and interferon regulatory factors, as well as the effector genes, for example 2′-5′-oligoadenylate synthetase and myxovirus proteins, are highly induced by the vaccine in a mammalian subject.

Even without the inclusion of transgenes that express mammalian immune factors, attenuated RV activates innate immune responses in the infected subject, including expression of IFN-α/β and chemokines (Example II). The induction of the innate immune responses therefore appears to play an important role in RV attenuation by preventing RV spread from the spinal cord to the brain. It is known that IFN-α/β and chemokines also enhance the adaptive immune responses. Therefore, attenuated rabies viruses of the invention that express one or more IFN-α/β and/or chemokines are expected to further attenuate RV as well as to induce innate and enhance the adaptive immune responses. The rabies vaccine of the invention is therefore expected not only to induce strong innate immune responses in a subject but may also enhance adaptive immune responses, such as the production of neutralizing antibodies. Neutralizing antibodies react with the rabies virus and destroy or inhibit its infectivity and virulence. The presence of a neutralizing antibody may be, for example, detected directly using immunohistochemical techniques, or demonstrated by means of mixing serum with the suspension of the virus, and then injecting the mixture into animals or cell cultures that are susceptible to virus in question. The vaccine preferably contains an amount of attenuated rabies virus effective to induce neutralizing antibodies in a subject. The amount of attenuated rabies virus in the vaccine is preferably effective to protect the subject against experimental or natural challenge with rabies virus. The vaccine may induce immune responses in the central nervous system (CNS) and help clear the virus from the CNS.

The rabies vaccines of the invention are readily formulated as pharmaceutical compositions for veterinary, wildlife management, or human use. The pharmaceutical composition optionally includes excipients or diluents that are pharmaceutically acceptable as carriers and compatible with the attenuated rabies virus. The term “pharmaceutically acceptable carrier” refers to a carrier(s) that is “acceptable” in the sense of being compatible with the other ingredients of a composition and not deleterious to the recipient thereof or to the live attenuated virus. Suitable excipients include, for example, water, saline, dextrose, glycerol, ethanol, or the like and combinations thereof. In addition, if desired, the vaccine may contain minor amounts of auxiliary substances such as wetting or emulsifying agents, pH buffering agents, salts, and/or adjuvants which enhance the effectiveness of the immune-stimulating composition. For oral administration, the vaccine can be mixed with proteins or oils of vegetable or animal origin, and may be presented in the form of a bait when administered in the wild. Methods of making and using such pharmaceutical compositions are also included in the invention.

The vaccine of the invention can be administered to any mammal including humans, domesticated animals (e.g., cats and dogs) and wild animals (e.g., stray dogs, foxes, raccoons and skunks).

Dosage amounts, schedules for vaccination and the like for the rabies vaccine of the invention are readily determinable by those of skill in the art. For example, Mebastion (U.S. Pat. No. 6,887,479) describes the administration of a live attenuated rabies vaccine to warm-blooded animals. The vaccine of the invention can be administered to the mammal using any convenient method, preferably parenterally (e.g., via intramuscular, intradermal, subcutaneous or intracranial injection) or via oral or nasal administration. The useful dosage to be administered will vary, depending on the type of mammal to be vaccinated, its age and weight, the immunogenicity of the attenuated virus, and mode of administration. In general a suitable dosage will vary between 10² to 10⁸ TCID₅₀/mammal. TCID₅₀ is 50% Tissue Culture Infective Dose.

The vaccine of the invention can be administered prophylactically, prior to exposure to the rabies virus, or it can be administered as a therapeutic after exposure to the virus. Therapeutic administration of the vaccine to infected subjects is effective to delay or prevent the entry of the rabies virus into the central nervous system or brain of the subject, and/or to kill or otherwise disable the rabies virus, or cells containing the rabies virus, in the subject. If administered after exposure to the rabies virus, it is preferably administered as soon after exposure as possible, prior to the appearance of symptoms. The vaccine may also be administered after appearance of symptoms. The vaccine may be used in people who have been exposed to rabies virus without administration of anti-rabies immunoglobulin. Prophylactic administration of vaccine to uninfected subjects is effective to reduce either or both of the morbidity and mortality associated with subsequent infection by rabies virus.

The present invention is illustrated by the following examples. It is to be understood that the particular examples, materials, amounts, and procedures are to be interpreted broadly in accordance with the scope and spirit of the invention as set forth herein.

### Examples

### Example I

### Mutated Rabies Virus

Mutation of the Nat the Phosphorylation Sites Reduced the Rate of RV Transcription and Replication, Consequently Leading to further Attenuation

RV N plays important roles in the regulation of viral transcription and regulation, and RV N is phosphorylated on the serine at position 389. Our studies demonstrated that mutation of the N at the phosphorylation site inhibited the rate of minigenome transcription and replication (Yang et al., 1999, J. Virol. 73:1661-1664). To determine if the same mutation can reduce the rate of viral replication in the full infectious virus, we mutated S389A, S389D, and S389E of the N on the full infectious clone L16 (Wu et al., J. Virol. 2002, 76:4153-4161). It was found that mutations from S to A and S to D resulted in reduction of both viral transcription and replication. Northern blot hybridization with RV probes revealed that the rate of viral transcription (mRNA) and replication (genomic RNA) was reduced by as much as 90%, particularly when the S was mutated to A. Growth curve studies indicated that production of the mutant virus with S to A mutation (L16A) was as much as 10,000-fold less than that of the parental virus. These studies indicate that mutation of the N at the phosphorylation site can reduce the rate of viral transcription and replication in the full infectious virus. Fu, U.S. Pat. No. 6,706,523, issued Mar. 16, 2004.

To determine if the recombinant RV is further attenuated than the parental virus, we inoculated adult mice by IC with either L16A or L16 at the doses of 10³, 10⁴, or 10⁵ ffu. Mice infected with the parental virus L16 all succumbed to rabies by day 10. None of the mice infected with L16A at the doses of 10³ and 10⁴ ffu developed disease. However, 50% of the mice infected with 10⁵ ffu of L16A developed rabies. These studies indicate that mutation of the N at the phosphorylation site further attenuated RV. To determine if the recombinant RV is stable, L16A was passaged in BSR cells for 20 passages, total RNA was prepared from infected cells every 5 passages for RT-PCR and sequencing, no reversion was detected during the 20 passages in BSR cells. Total RNA was also prepared from the brains of mice died of infection with L16A. RT-PCR and sequence analyses indicate the recombinant virus from the sick animals reverted to the parental virus at the mutation site. This study indicates that the mutant virus with S389A is almost avirulent. However, it reverted under pressure in vivo. Most likely, this is because only one nucleotide was changed when the S (TCT) was mutated to A (GCT).

### Construction of Double Mutant Virus

To reduce the possibility of reversion, we mutated the S at 389 to glycine (G, GGT), asparagines (N, ATT), and glutamine (Q, TTG). Initially we tested the effect of these mutations on viral replication in RV minigenome using CAT assay. It was found that mutation from S to G, N, or Q resulted in reduction of viral replication more than the mutation from S to A, D, or E, respectively. Furthermore, mutation from S to G, N and Q resulted in change of at least two nucleotides, thus making the virus less likely to revert. To determine if mutation from S to G, N, and Q in the full-infectious virus will results in more RV attenuation, we mutated the S at 389 to G, N, Q, respectively. These full infectious clones were designated pL16G, pL16N, and pL16Q, respectively.

Construction offull infectious clone of RV with mutation on both the N and G Previous studies showed that mutation of the G at R333 leads to attenuation by reducing its neuroinvasiveness (Flamand et al. “Avirulent mutants of RV and their use as live vaccine.” Trends. Microbiol. 1993;1:317-320; Lafay et al. “Vaccination against rabies: construction and characterization of SAG-2, a double avirulent derivative of SAD Bern.” Vaccine 1994;12:317-320). We hypothesize that mutation on both the N (S389) and G (R333) will reduce not only neuroinvasiveness, but also the rate of viral replication. To construct RV with double mutations, we used pL16, pL16G, pL16N, and pL16Q as templates. To incorporate the R333 mutation into each of the infectious clones, we subcloned an XhoI fragment containing the R333 of the G and the R (AGA) was mutated to glutamic acid (E, GAA) using primers (5′ATGCTCACTACAAGTAAACTTGGAATCAG3′ and 5′GGAGGATCTCATTCCAAGTTTCACTTGTAG3′; SEQ ID NOs:1 and 2, respectively). This R to E mutation has been shown to make RV avirulent (Seif et al. “Rabies virulence: effect on virulence and sequence characterization of RV mutations affecting antigenic site III of the glycoprotein.” J. Virol. 1985;53:926-934.). Furthermore, two nucleotides were changed, thus reducing the possibility for the virus to revert. The mutated XhoI fragments were then cloned back to the respective plasmids, creating infectious clones pL16-G, pL16G-G, pL16N-G, and pL16Q-G.

### Construction of Recombinant RV by Relocating the G Gene

As we have shown that mutation of the N at the phosphorylation site reduced the rate of replication, it is possible that these mutated RVs could have reduced immunogenicity despite the fact that they are avirulent. To increase the immunogenicity of these avirulent viruses, we propose to relocate the G from the 4^{th} to the 1^{st} position. RV G is the only surface G that stimulates the production of VNA that provide protection. Like many of the negative-stranded and non-segmented RNA viruses, RV transcription attenuated at each of the gene junctions. Thus relocation of the G from the 4^{th} to the 1^{st} position will increase the G expression.

To relocate RV G from the 4^{th} to the 1^{st} position, plasmid pL16-G was digested with Xhol. Since relocation of the G on VSV from the 4^{th} to the 1^{st} position still caused disease in mice and pigs, we relocated the G on infectious clone, L16-G (avirulent, essentially SAG2). The large fragment (10.5 kb) was self-ligated to form the plasmid pL16ΔXhoI, which contains the N, P, M, and partial G and L genes. The small fragment (4.5 kb, 3854-8273 of the RV genome) was cloned into pGEM-3Z vector at the XhoI site and designated pGEM-XhoI. The plasmid pL16ΔXho was used as a template for PCR to delete the N, P, and M genes, using PfuTurbo Hotstart DNA polymerase (Stratagene) and primers (5′AACATCCCTCAAAAGACTCAAGG3′ and 5′ACATTTTTGCTTTGCAATTGACAATGTC3′; SEQ ID NOs:3 and 4, respectively). PfuTurbo Hotstart DNA polymerase creates blunt ends in the amplified fragments that can be self-ligated. The ligated plasmid was sequenced at the mutation junction and there was no spurious change in leader sequence and the starting sequence for the G gene. The resulting plasmid was designed pSAD-GL. In order to insert N-P-M genes between G and L, we created a unique Hpal site between the G and L genes, immediately after the intergenic sequence of the G+ψ. This mutation was introduced into the gene on plasmid pGEM-XhoI because this plasmid contains the intergenic sequence between the G and L. Site directed mutagenesis was performed using primers (5′CAGAAGAACAACTGTTAACACTTCTC3′ and 5′GAGAAGTGTTAACAGTTGTTGTTCTTCTG3′; SEQ ID NOs:5 and 6, respectively) to insert the HpaI site. The mutated plasmid was designated pGEM-GHL and was used to insert N-P-M sequence amplified from pL16ΔXhoI using primers (5′AACACCCCTACAATGGATGCCG3′ and 5′AATAGTTTTTTTCACATCCAAGAGG3′; SEQ ID NOs:7 and 8, respectively). After confirming the orientation, the plasmid was designated pGEM-GNMPL. Finally, the XhoI fragment from pGEM-GNMPL was cloned into pSAD-GL to create the full-length RV clone with the G gene relocated to the 1st position. This plasmid was designated pG1N2. Some of the recombinant RVs (L16-G, L16N, L16Q, and L16Q-G) have been selected. L16-G is essentially SAG2. Both viruses are derived from SAD-B19 and bear the R333E mutation on the G. The only difference is that L16-G is made from an infectious clone while SAG2 is selected with neutralizing antibodies.

Identification of Cellular Casein Kinase II (CK-II) as the Kinase that Phosphorylates RV N

Since we demonstrated that mutation of the N at the phosphorylation site resulted in reduction of RV replication and RV attenuation, we identified the kinase that phosphorylates RV N. N expressed alone in mammalian and insect cells is phosphorylated, suggesting that it is cellular kinase that phosphorylates RV N. Because the phosphorylation site at 389 (SDDE) of RV N resembles the CK-II motif (SX.XD/E), it is possible that CK-II phosphorylates RV N. To test this hypothesis, we expressed the N in E. coli and purified it by metal affinity chromatography. The recombinant N was phosphorylated by BHK cellular extracts and by purified CK-II. In addition, the phosphorylation of the recombinant N in vitro can be blocked by a CK-II inhibitor, heparin. Furthermore, N phosphorylation in the virus-infected cells can be inhibited by a CK-II specific inhibitor, 5,6-dichloro-β-D-ribofuranosyl benzimidazole. However, PKC did not phosphorylate the recombinant N in vitro; nor did staurosporine, a PKC and other kinase inhibitor, prevented N from phosphorylation in the virus-infected cells. Thus, our data demonstrate that cellular CK-II phosphorylates RV N (Wu et al., Biochem. Biophysic. Res. Comm., 2003, 304:333-338).

N Phosphorylation Occurs after RNA Encapsidation

To understand the mechanism by which N phosphorylation reduces viral transcription and replication, we investigated at what stage of the virus infectious cycle, RV N is phosphorylated. Since N phosphorylation is involved in the N-P and N-RNA interactions, we determined the pattern of N phosphorylation when N is expressed alone, N and P are co-expressed, or when N and P are co-expressed together with minigenomic RNA. We found that RV N is phosphorylated only when it is bound to RNA. When the N and P are co-expressed without the presence of the genomic RNA, the N in the N-P complex is not phosphorylated. Thus our results indicate that N phosphorylation does not play a role in the RNA encapsidation process per se. Rather the negatively charged phosphoserine of the N and the negatively charged genomic RNA may weaken the interaction between N and RNA in the RNP complexes, thus facilitating the initiation of next round viral RNA transcription and replication (Liu et al., 2004, J. Gen. Virol. 85:3725-3734).

### Example II

### Attenuated Rabies Virus Activates, While Pathogenic Rabies Virus Evades, the Host Innate Immune Responses in the CNS

Rabies virus (RV) induces encephalomyelitis in humans and animals. However, the pathogenic mechanism of rabies is not fully understood. To investigate the host responses to RV infection, we examined and compared the pathology, particularly the inflammatory responses, and the gene expression profiles in the brains of mice infected with wild-type (wt) virus SHBRV or laboratory-adapted virus B2C, using a mouse genomic array. An oligonucleotide microarray (Affymetrix Mouse Expression Set MOE430A) and real-time PCR were used to identify candidate genes that are differentially expressed in the CNS of mice infected with the pathogenic SHBRV or the attenuated B2C.

Extensive inflammatory responses were observed in animals infected with the attenuated RV, but little or no inflammatory responses were found in mice infected with wt RV. Furthermore, attenuated RV induced the expression of the genes involved in the innate immune and anti-viral responses, especially those related to the IFN-α/β signaling pathways and inflammatory chemokines. For the IFN-α/β signaling pathways, many of the interferon regulatory genes such as the signal transduction activation transducers (STAT), interferon regulatory factors (IRF), as well as the effector genes, for example, 2′-5′ oligoadenylate synthetase (OAS) and myxovirus proteins (Mx), are highly induced in mice infected with attenuated RV. However, many of these genes were not up-regulated in mice infected with wt SHBRV. The data obtained by microarray analysis were confirmed by real-time PCR. Together, these data suggest that attenuated RV activates, while pathogenic RV evades, the host innate immune and anti-viral responses. The attenuated RV was found to be a potent activator of the host innate immune system, particularly the IFN-α/β signaling pathway and inflammatory reaction, whereas the pathogenic SHBRV was a poor inducer of the innate immune responses. Thus, evasion of the innate immune responses may be one of the mechanisms by which wt SHBRV contributes to its pathogenicity and neuroinvasiveness. Wang et al., 2005, J. Virol. 79:12554-12565.

### Materials and Methods

Animals, viruses, and antibodies: Female ICR mice (Harlan) at the age of 4-6 weeks were housed in temperature- and light-controlled quarters in the Animal Facility, College of Veterinary Medicine, University of Georgia. They had access to food and water ad libitum. Two RV strains were selected for this study. One is SHBRV, a wt RV isolated from a human patient (Morimoto et al., 1996, Proc Natl Acad Sci USA, 93:5653-8), and the other CVS-B2C, a laboratory-adapted, attenuated virus isolated from CVS-24 virus by passaging in BHK cells (Morimoto et al., 2000, J Neurovirol. 6:373-81). Virus stocks were prepared as described (Tuffereau et al., 1998. EMBO J. 17:7250-9).

Briefly, one-day-old suckling mice were infected with 10 μl of viral samples by the intracerebral (IC) route. When moribund, mice were euthanized and brains were removed. A 10% (w/v) suspension was prepared by homogenizing the brain in Dulbecco's modified Eagle's medium (DMEM). The homogenate was centrifuged to remove debris and the supernatant collected and stored at −80° C. Anti-RV nucleoprotein (N) monoclonal antibody 802-2 (Galelli et al., 2000, J Neurovirol. 6:359-72), was obtained from Dr. Charles Rupprecht, Center for Disease Control and Prevention. Anti-RV glycoprotein (G) polyclonal antibody was prepared in rabbit as described (Faber et al., 2004, Proc. Natl. Acad. Sci. U S A, 101:16328-32), and has been shown to have similar affinity to the G from wt SHBRV and laboratory adapted CVS-N2C (Yan et al., 2001, J Neurovirol 7: 518-527). Antibodies to STAT1, STAT2, and STAT3 were obtained from Chemicon International Inc. Anti-CD3 polyclonal antibody was purchased from Abcam, England.

Mouse primary neuronal cultures. Mouse primary neuronal cultures were prepared using standardized procedures as described (Adamec et al., 2001, Brain Res. Protocol 7:193-202; Kiecolt-Glaser et al., 2003, Proc Natl Acad Sci U S A 100:9090-5). Swiss-Webster mice at gestation day 16 were euthanized and the embryos removed. Neocortex from these embryos were collected and digested with trypsin. Separated neuronal cells were then plated into culture wells treated with poly-D-lysine (50 μg/ml). The primary neurons were grown in MEM medium in a humidified atmosphere of 5% CO₂-95% air at 37° C. Ara-c (cytosine furo-arabinoside) at 1 μM final concentration was added at 3 to 5 days after plating to prevent the proliferation of non-neuronal cells.

Animal infection and tissue collection. Mice were infected with 10 ICLD₅₀ of either virus (B2C or SHBRV) by the IC route. Alternatively, mice were infected with 10 IMLD50 by the intramuscular (IM) route in the hind legs (both sides). Infected animals were observed twice daily for 20 days for the development of rabies. Sham-infected mice were included as controls. At the time of severe paralysis, mice were sacrificed and brains removed and flash-frozen on dry ice before being stored at −80° C. For histopathology and immunohistochemistry, animals were anesthetized with ketamine/xylazine at a dose of 0.2 ml and then perfused by intracardiac injection of PBS followed by 10% neutral buffered formalin as described (Yan et al., 2001, J. Neurovirol 7:518-527). Brain tissues were removed and paraffin embedded for coronal sections (4 μm).

Total RNA extraction. Mouse brain (400-500 mg each) was homogenized in 3 ml TRIZOL (Invitrogen-Life Technologies). Total RNA was extracted and purified using RNeasy Mini Kit (Qiagen) following the manufacturer's specifications.

Microarray hybridization and analysis. cRNA used for microarray hybridization was prepared following Affymetrix Eukaryotic Sample and Array Processing protocol, and then hybridized to Affymetrix mouse expression microarray (Mouse Expression Set MOE430A). Eight pg total RNA was used in the first strand cDNA synthesis, together with T7-(dT)24 primer and Superscript II reverse transcriptase (Invitrogen-Life Technology). Second strand cDNA was synthesized using E. coli DNA ligase, DNA polymerase I, RNase H and T4 DNA polymerase (Invitrogen-Life Technologies), and then purified using the GeneChip Sample Cleanup Module (Affymetrix). Biotin-labeled cRNA was prepared by using Enzo RNA Transcript Labeling Kit (Affymetrix), and then purified by GeneChip Sample Cleanup Module. cRNA was fragmented and spiked with bacterial control genes (bioB, bioC, bioD, and cre) before overnight hybridization to Affymetrix mouse MOE430A. The hybridized microarrays were washed by using GeneChip Fluidics Station, and then stained with R-phycoerythrin-straptavidin using Antibody Amplification Washing and Staining Protocol. GeneArray scanner was used to scan the hybridized gene chip, GeneChip Operating Software (GCOS) was used to collect data, and Statistical Expression Algorithm was used to obtain the signal values. Signals were scaled to a target intensity of 500 for normalization. Genes that are differentially expressed (at least 2 fold) were used in hierarchical analysis by dChip developed by the Wong Lab, Department of Biostatistics, Harvard School of Public Health (http://biosun1.harvard.edu/complab/dchip/install.htm). Analysis of gene pathways was carried out by using gene ontology from the GO Consortium (http://www.geneontology.org/GO.consortiumlist.shtml).

Real-time SYBR Green PCR: To confirm the data generated from the microarray, real-time PCR was performed on the RNA samples using gene specific primers in a Stratagene Mx3000P instrument. PCR reaction was performed in one step in 25 μl volume, with 100 ng sample RNA. Each reaction was carried out in duplicate. The reverse transcriptase and DNA polymerase were from Brilliant SYBR Green QRT-PCR Master Mix Kit (Stratagene). cDNA synthesis was performed at 50° C. for 30 min. During quantitative analysis, standard curves of three points were used to calculate amplification efficiency for each pair of primers. GAPDH was used as an endogenous reference gene.

Histopathology, immunohistochemistry, and Western-blotting. Histopathology was performed by staining the paraffin embedded sections with hematoxylin and eosin (H&E). For immunohistochemistry, paraffin-embedded brain sections were heated at 70° C. for 10 minutes, then dipped in CitriSolv (Fisher Scientific) for 3×5 min and dried until chalky white. Slides were incubated with proteinase K (20 μg/ml) in 10 mM TrisHCL (pH 7.4-8.0) for 15 min at 37° C. and rinsed three times with PBS. The primary antibody used was either the monoclonal antibody 802-2 directed against RV N (Hamir et al., 1995, Vet. Rec. 136:295-296), the rabbit polyclonal anti-RV G antibody (Fu et al., 1993, Vaccine 11:925-928), or anti-CD3 polyclonal antibody. The secondary antibodies used were biotinylated goat anti-mouse or goat anti-rabbit IgG. The avidin-biotin-peroxidase complex (ABC) was then used to localize the biotinylated antibody. Finally diaminobenzidine (DAB) was used as a substrate for color development. For Western blotting, brain extract as well as cell extract were subjected to electrophoresis on a 10% polyacrylamide SDS gel. After separation on SDS-PAGE, proteins were electroblotted to PDVF membranes. Blots were then blocked in PBS containing 5% nonfat milk and 0.05% Tween-20 for 1 hr at room temperature with shaking. Then, blots were incubated with the respective antibodies overnight at 4° C. or for 2 hr at room temperature. After three washes with PBS containing 0.05% Tween-20 (PBST), blots were incubated for 1 hr with HRP-conjugated secondary antibody, followed by extensive washes in PBST. Proteins were detected by enhanced chemiluminescence (ECL, Amersham Biosciences). Band signals corresponding to immunoreactive proteins were measured and scanned by image densitometry using Adobe Photoshop 6.0 software.

Immunofluorescence and confocal microscopy. Primary neurons grown on coverslips were infected with each of the viruses and then fixed with 4% paraformaldehyde at day 5 after infection. Viral antigens were detected by using FITC-conjugated with anti-RV N monoclonal antibodies (Centocor, Pa.). The expression of STAT1, STAT2, and STAT3 was detected by using rabbit anti-STAT polyclonal Abs (Chemicon). Anti-rabbit secondary antibody conjugated with Alexa 488 (Molecular Probes) was used for 1 hour at room temperature. Propidium Iodide (PI) was used for counter staining (15 min, RT). After washing, the coverslips were mounted with aqueous anti-fade mounting medium and examined under a Leica TCS NT confocal microscope. The percentage of cells with nuclear translocation of STAT proteins was evaluated by counting six areas and the average number of translocated cells was calculated.

### Results

SHBRV is more Pathogenic, but Induces Fewer Inflammatory Changes, than B2C

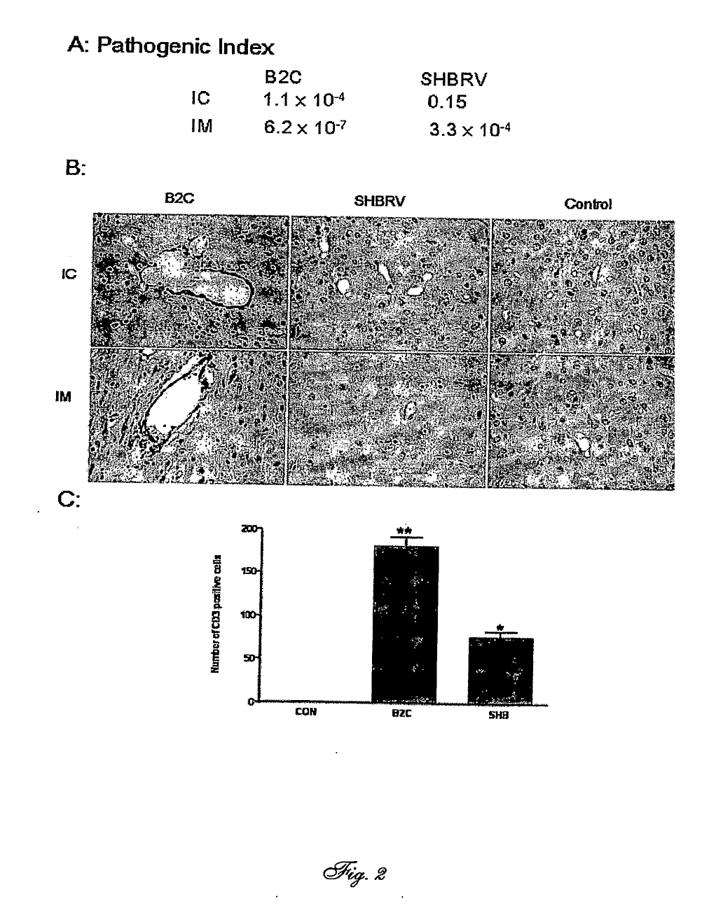

To compare the pathogenicity of these two viruses, SHBRV and B2C, the IC and IM pathogenic indices were determined (Li et al., 2005, J. Virol. 79:10063-10068; Morimoto et al., 2000, J. Neurovirol. 6:373-81; Morimoto et al., 1998, Proc. Natl. Acad. Sci. USA., 95:3152-3156), by subtracting the log virus titer/ml in BHK cells from the log ICLD₅₀/ml or the log IMLD₅₀/ml and the results are shown in FIG. 2A. Almost 1000 times more viral particles are required for attenuated B2C than for SHBRV to kill infected mice by either the IC or the IM route, suggesting that SHBRV is more pathogenic than B2C in the mouse model.

Although it has been previously reported that very little inflammation and neuronal loss was observed in rabies patients (Murphy. 1977, Arch. Virol., 54:279-297), laboratory-adapted viruses induced extensive inflammation and necrosis (Miyamoto et al., 1967, J. Exp. Med., 125:447-456; Yan et al., 2001, J. Neurovirol 7:518-527). To examine the pathological changes in mice infected with each virus, mice were transcardially perfused and brains removed for histopathology and immunohistochemistry. It was found that attenuated B2C induced extensive pathological changes, particularly inflammation including perivascular cuffing, gliosis, and infiltration of macrophages and lymphocytes. Necrosis and apoptosis were also observed frequently in brain tissues infected with B2C. On the other hand, only a few histological changes were observed in mice infected with SHBRV by the IC or IM routes (FIG. 2B). To quantify the inflammatory reactions, CD3-positive cells were measured using anti-CD3 antibodies in the cortex in mice infected by the IC route as described previously (Li et al., 2005, J. Virol. 79:10063-10068). Three serial sections were selected from each animal for measurement and the average number of CD3-positive cells was obtained and analyzed for statistical significance by one way ANOVA. As shown in FIG. 2C, significantly (p<0.01) more CD3-positive cells were detected in B2C- than in SHBRV-infected mice, indicating that more inflammatory cells infiltrated B2C-infected than SBBRV-infected mouse brain.

Pathogenic SHBRV Induces Fewer Changes in Host Gene Expression than B2C

To investigate the different host responses to infection with the attenuated and the wt RV, mice were infected IC with 10 ICLD₅₀ of the pathogenic SHBRV or the laboratory adapted B2C. Alternatively, mice were infected IM with 10 IMLD₅₀ of each virus. Sham-infected mice were used as controls. Mice were sacrificed when developing severe paralysis and flash frozen brains were used for total RNA extraction and cRNA synthesis. The cRNA was then used to hybridize to the mouse whole genomic microarray, mouse expression set 430A. The data were analyzed by a combination of GCOS and dChip. The normalized data for 22,626 mouse genes were collected. Changes over two-fold are considered for either up- or down-regulation. When compared with controls, there are 792 genes up-regulated and 301 genes down-regulated in animals infected IC with B2C, while there are 525 genes up-regulated and 107 genes down-regulated in animals infected IC with SHBRV. In comparison, there are 890 genes up-regulated and 694 genes down-regulated in animals infected IM with B2C, while there are 259 genes up-regulated and 198 genes down-regulated in animals infected IM with SHBRV. Overall, pathogenic SHBRV induced fewer changes in host gene expression than B2C in either IM or IC infected mice. Although there is a number of genes whose expression is altered by one virus infection, but not by the other, there are very few genes whose expression is up-regulated by one virus and down-regulated by another. Table I compares the numbers of the genes up- and/or down-regulated in animals infected with each virus and by each route.

### Attenuated B2C, but not wt SHBRV, Activates the Gene Expression of the Innate Immune Responses

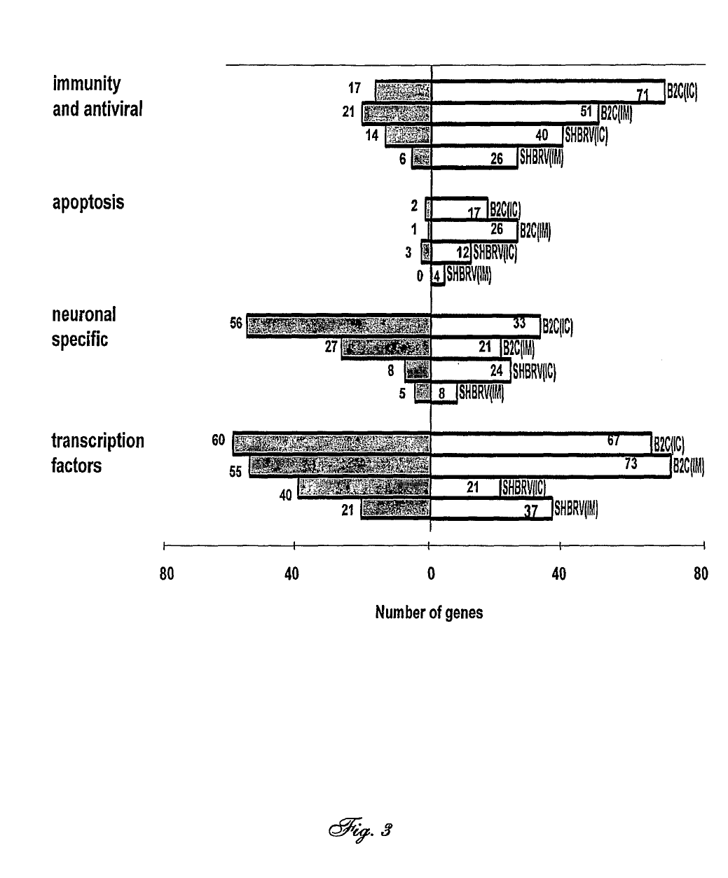

Analysis of the microarray data by gene ontology revealed that pathogenic and attenuated RVs differentially induce host gene expression in many of the gene clusters (FIG. 3). For immunity and antiviral genes as well as genes involved in apoptosis, there are more genes up- than down-regulated in mice infected with either virus. In addition, there are more genes up-regulated in mice infected with B2C than with SHBRV. For genes involved in neuronal functions, there are more genes down-than up-regulated, particularly in mice infected with B2C. For transcription factors, the numbers of genes up-regulated and down-regulated are similar for B2C by each route of infection. In this paper, only the genes involved in the innate immune and anti-viral responses are analyzed in detail, particularly those up-regulated. The modification of other host genes will be described in more detail elsewhere.

Analysis of the gene profiles involved in the innate immune and anti-viral responses revealed that the attenuated B2C (by either IC or IM inoculation) induced the expression of genes important in the innate immune responses, particularly the interferon (IFN)-α/β induction and IFN-α/β signaling pathway. Genes encoding inflammatory cytokines and chemokines are also up-regulated by infection with B2C. On the other hand, wt SHBRV is a poor inducer of the innate immune responses (FIG. 3). Many of the genes important for the immune and anti-viral responses are not up-regulated in SHBRV-infected animals. For those genes up-regulated by both virus infections, usually the increase is 2 to 30-fold higher in animals infected with B2C than with SHBRV (Tables 2 and 3).

In mice infected with B2C by the IC or IM routes, most of the genes involved in IFN-α/β pathway are up-regulated. These include IFN-α/β genes, genes involved in the IFN-α/β mediated signaling and transcription activation, and genes encoding proteins implicated in the anti-viral activities (see Table 2). Up-regulated IFN genes include IFN-α2, IFN-α4, and IFN-α5 as well as IFN-β. Interferon signaling genes (Cbp/p300-interacting transactivator, Stat1, Stat2, Stat3, and Jak-2) and interferon regulatory factors (IRF-1, 2, and 7) are up-regulated. IFN-α/β-induced proteins implicated in the anti-viral activities, including double-stranded RNA-dependent protein kinase (PKR), the 2′,5′-oligoadenylate synthetases (OAS), RNA-specific adenosine deaminase (ADAR), myxovirus resistance (Mx), and major histocompatability (MHC) class I are also up-regulated in B2C-infected animals. The up-regulated genes for 2′5′-OAS include OAS-1B, 1G, 2, and 3 as well as OAS-like 1 and 2. Along the IFN signaling pathway, many of the IFN-activated and inducible genes are highly up-regulated (IFN activated gene 202B, 203, 204, and 205, IFN -induced transmembrane protein with tetratricopeptide repeats 1, 2, and 3). The most up-regulated gene is the anti-viral Mxl that is increased 388 fold in animals infected with B2C by the IC route.

On the other hand, many of genes important in the IFN-α/β pathway are not up-regulated in SHBRV-infected mice. The IFN genes are not up-regulated except IFN-α4 (6 fold) by IC and IFN-β (2 fold) by IM. For the IFN signaling and effector genes, Cbp/p300-interacting transactivator, Stat3, Jak-2, IRF-2, 2′5′-OAS-2, 2′5′-OAS-3, ADAR, MHC-1, PKR, IFN activated gene 203, and IFN -induced transmembrane protein with tetratricopeptide repeats 3 are not up-regulated in mice infected with SHBRV by the IC or the IM route. Some of the genes in the IFN-α/β pathway are up-regulated in mice infected with SHBRV but the increase was 2 to 30-fold lower than that in mice infected with B2C (Table 2).

Components in the inflammatory pathway including toll-like receptors (TLR), chemokines, cytokines, and complement components are also up-regulated in B2C-infected animals (Table 3). The expression of TLR1, TLR2, and TLR3 is up-regulated. Pro-inflammatory chemokines in both the C-C and C-X-C families including Rantes (CCL5), MCP-1 (CCL2), MCP-3 (CCL7), and MCP-5 (CCL12), MTP-1α. (CCL3), MIP-1β (CCL4), MIP-2α (CXCL-1), and MIP-2β (CXCL-2), and IP-10 (CXCL-10) are all up-regulated with some increased more than 100 fold. Many of the cytokines and cytokine receptors are up-regulated, for example, the pro-inflammatory cytokine IL-6. Complement components, such as c1q, c1r, c1s, c2, c3 and c4, are up-regulated. In mice infected with SHBRV, TLR1 and TLR2 are not up-regulated in mice infected by the IC or IM routes. For chemokines, only MCP-5 is up-regulated in SHBRV-infected mice to a similar level as in B2C-infected animals. MIP-1α, MIP-1β, and CXC chemokine BLC are not up-regulated in mice infected with SHBRV by the IC or IM routes. The up-regulation of other chemokines in mice infected with SHBRV is 2 to 20-fold lower than that in mice infected with B2C (Table 3). Likewise, expression of many cytokine, cytokine receptors, and complements is not up-regulated in mice infected with SHBRV.

### Confirmation of Microarray Data by Real-Time PCR.

To validate the microarray data, real-time PCR was performed on selected genes from each of the categories including IFN (IFNα-2 and IFNα-5), IFN regulatory genes (Stat1, Stat2, Stat3, IRF2, and IRF7), IFN-effector genes (OAS-1G and Mx1), and chemokine genes (MCP-1, IP-10, and Rantes). GAPDH was used as a reference gene. Primers for amplification of these genes are listed in Table 4. The results from the real-time PCR were compared with the data obtained by microarray hybridization and summarized in Table 5. The fold increases in mice infected with either SHBRV or B2C over the controls are similar for some genes in both microarray data and real-time PCR results. For other genes such as Stat1, Stat2, OAS-1G, Mx1, IP-10, and Rantes, real-time PCR is more sensitive and detected greater increases than the microarray hybridization. Nevertheless, the ratios between B2C and SHBRV in fold increases are similar in both the microarray and the real-time PCR.

### The Increased Expression of Stat Genes Resulted in Increased Synthesis of STAT Proteins

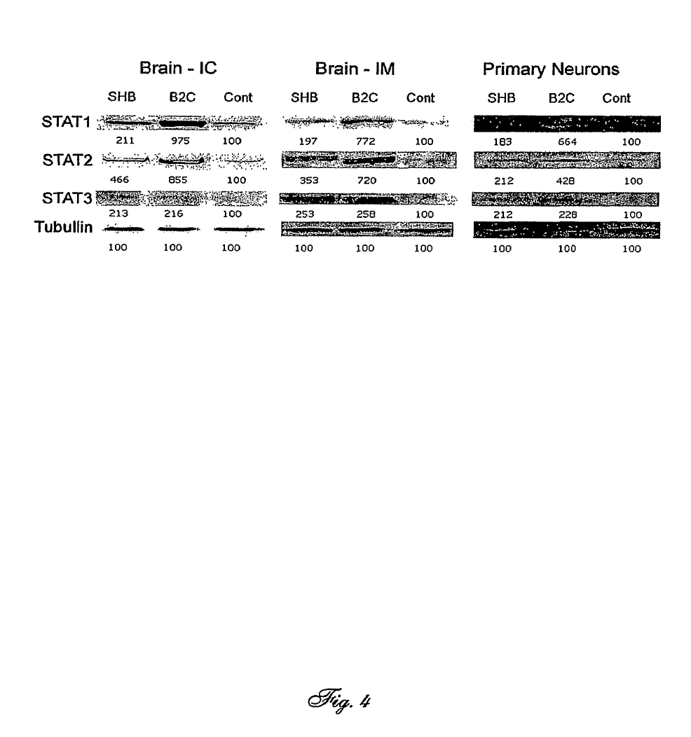

To determine if increased Stat gene expression results in increased protein synthesis, the levels of STAT1, STAT2, and STAT3 (protein yield) in the IC- or IM-infected mice were measured by Western blotting using anti-STAT antibodies and the band intensity was measured by densitometry. As shown in FIG. 4, the expression of STAT1 and STAT2 increased more than 7 fold in mice infected with B2C by either the IC or the IM route, whereas STAT1 and STAT2 increased only 2 to 4 fold in mice infected with SHBRV. On the other hand, STAT3 expression is up-regulated similarly in animals infected with each of the viruses and its level increased about 2 fold over the controls. The levels of increased STAT proteins are proportional to that of increased Stat transcripts as detected by microarray and real-time PCR (Tables 2 and 5). The level of β-tubulin expression is almost the same in infected or uninfected animals. To further determine the expression pattern of the STATs, primary neurons were infected with each virus at MOI of 0.1 and the cells were harvested at day 5 for Western blotting. The level of STAT1 and STAT2 increased 4 to 7 fold in cells infected with B2C, but only about 2 fold in cells infected with SHBRV. Likewise, the level of STAT3 increased about 2 fold in cells infected either with B2C or SHBRV. These data indicate that the increased expression of Stat genes resulted in increased STAT protein synthesis.

### STAT1 and STAT2, but not STAT3, are Activated by RV Infections.

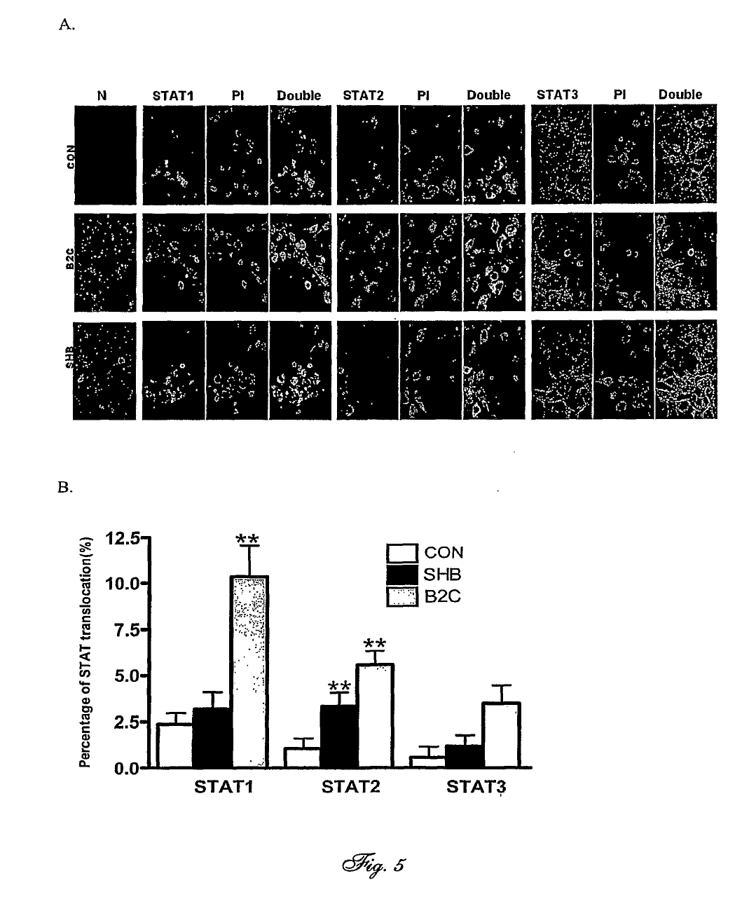

STATs, particularly STAT1 and STAT2, play an important role in the IFN-α/β signaling pathway. IFN-α/β binds to IFN-α/β receptors, which activates STATs by phosphorylation (Stark et al., 1998, Annu Rev Biochem. 67:277-64) Phosphorylated STATs form specific multimeric complexes that then translocate to the nucleus and initiate transcription (Darnell et al., 1994, Science, 264:1415-21). To determine if STATs are activated by RV infection, neurons infected with B2C or SHBRV were fixed with 4% paraformaldehyde and subjected to immunocytochemistry with anti-STAT antibodies and confocal microscopy. The percentage of cells with nuclear translocation was quantified in neurons at 1, 3, and 5 days p.i. There were only a few translocated cells for any of the STAT proteins at 1 or 3 days p.i. in cells infected with either virus. As shown in FIG. 5, significantly more cells with nuclear translocation were observed for STAT1 and STAT2 in cells infected with B2C at day 5 p.i. Only a few cells with nuclear translocation were detected for STAT3 in cells infected with B2C. In addition, significant more cells with nuclear translocation for STAT1 and STAT2 proteins were observed in cells infected with B2C than with SHBRV. Together, these data suggest that STAT1 and STAT2, but not STAT3, are involved in the IFN activation and effector pathway in RV infections.

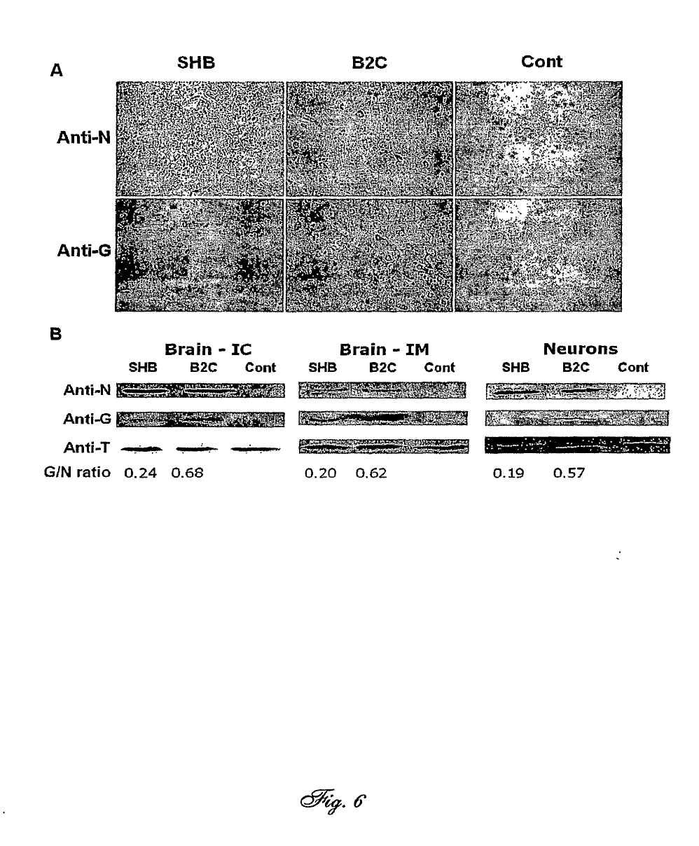

Evasion of the innate immune responses by pathogenic SHBRV correlates with the restriction of RV G expression: Previously, it has been reported for pathogenic RV that restriction of the G expression of contributes to its pathogenicity (Morimoto et al., 1999, J. Virol. 73:510-8; Yan et al., 2001, J. Neurovirol. 7:518-527). To determine if restriction of G expression also occurs in pathogenic SHBRV infections and might correlate with the evasion of the innate immune responses by pathogenic SHBRV, the expression of RV G and N was evaluated on brain sections by using immunohistochemistry. As shown in FIG. 6A, the levels of N expression are similar in mice infected IC with each virus whereas the level of G expression is almost undetectable in SHBRV-infected mice. In contrast, the level of G expression is high in B2C-infected mice. To confirm this finding, brain extracts were made from mice infected with SHBRV or B2C by either the IC or IM routes. Furthermore, cellular extracts were prepared from primary neurons infected with each virus. All these extracts were used for Western blotting to detect the level of G and N expression. As shown in FIG. 6B, the level of G expression is consistently three-fold lower in both animals and cells infected with SHBRV than with B2C while the levels of N expression are similar in animals and cells infected with either virus. These data suggest that G expression is consistently inhibited in pathogenic RV infections in vivo as well as in vitro and as a result the restriction of G expression may be one of the mechanisms by which pathogenic SHBRV evades the activation of the innate immune responses.

### Discussion

Host innate immune responses are the first line of defense against infections. During the pathogen-host co-evolution, many viruses have developed ways to evade the host innate immune responses, particularly the IFN pathways (Samuel, 2001, Clin. Microbiol Rev. 14:778-809). Previously, various groups have reported that RV infection can activate the innate immune responses. For example, RV infection triggers the expression of inflammatory cytokines (Baloul et al., 2004. J. Neurovirol. 10:372-82; Baloul et al., 2003, Biochimie 85:777-88), and chemokines (Galelli et al., 2000, J. Neurovirol. 6:359-72; Nakamichi et al., 2004, J. Virol. 78:9376-88), in vitro or in vivo. In all these studies, only laboratory adapted RV were used. In the present paper, we compared a laboratory-adapted and attenuated RV with a wt pathogenic RV and found for the first time that the pathogenic RV evades, while the attenuated RV activates, the host innate immune responses. As detected by the microarray technology and real-time PCR, almost all the genes involved in the activation of the IFN-α/β pathway and many of the inflammatory chemokines and cytokines are up-regulated in animals infected with attenuated RV B2C by either the IC or the IM routes. However, many of these genes are not up-regulated in animals infected with pathogenic SHBRV. For those genes involved in the IFN-α/β pathway that are up-regulated in SHBRV-infected animals, the magnitude of increase is at least 2 to 30-fold lower than that in B2C-infected mice. Furthermore, attenuated RV induces extensive CNS inflammation while pathogenic RV does not.

The attenuated B2C activates the innate immune responses, particularly the IFN-α/β signaling pathway. Recently, Nakamichi et al. (J. Virol. 78:9376-88), also reported the up-regulation of IFN-α/β in RAW macrophages after stimulation with laboratory adapted, attenuated RV. In a companion paper (Préhaud et al., 2005. J. Virol. 79:12893-12904) as well as a paper published very recently (Conzelmann, 2005, J. Virol. 79:5241-5248), RV infection induces the expression of IFN-β. In our present study, not only IFN-β, but also IFN-α2, 4, and 5, are found to be up-regulated by infection with attenuated RV: Furthermore, those genes that are involved in IFN-mediated signaling and transcription activation of cellular gene expression are up-regulated. These include interferon signaling genes (Stat-1, 2, 3, and Jak-2) and interferon regulatory factors (IRF-1, 2, and 7). As summarized by Samuel (Clin. Microbiol Rev. 14:778-809, 2001), IFN-α/β-induced proteins implicated in the anti-viral activities include PKR, the 2′,5′-OAS, ADAR, Mx, and MHC class I. Genes encoding these proteins are all up-regulated in B2C-infected animals. These molecules are involved in mRNA translation inhibition, RNA degradation, RNA editing, and CTL responses. In the IFN signaling pathway, many of the IFN-activated and inducible genes are highly up-regulated (IFN activated gene 202B, 203, 204, and 205, IFN -induced transmembrane protein with tetratricopeptide repeats 1, 2, and 3). Thus attenuated RV activated the IFN-α/β pathway. The role of IFN-α/β in resisting RV infection has previously been investigated. Direct administration of INF-α/β or IFN-inducing poly I:C resulted in various degree of protection against RV infection in mice, hamsters, rabbits or monkeys (Harmon et al., 1974, Antimicrob Agents Chemother. 6:507-11; Hilfenhaus et al., 1975, Infect. Immun. 11:1156-8), Hooper et al. (J. Virol. 72:3711-9, 1998), reported that higher virus titers were detected in IFN-α/β receptor knockout (IFNAR^{-/-}) mice than immunologically intact mice when infected with an attenuated CVS-F3. It also took longer time for the IFNAR^{-/-} mice (21 days) than normal counterparts (8 days) to clear the virus from the CNS. In addition, fully immunocompetent mice developed higher levels of virus neutralization antibodies than IFNAR^{-/-} mice. All these data indicate that IFN-α/β plays a role in RV resistance through both innate and adaptive immune responses.

In addition to the IFN-α/β pathway, attenuated RV also stimulates the expression of many genes encoding inflammatory molecules such as chemokines, cytokines, TLRs, and complements. Inflammatory cytokine IL-6 (Kiecolt-Glaser et al., 2003, Proc. Natl. Acad. Sci. USA. 100:9090-5), is highly up-regulated in B2C-infected animals. Many of the inflammatory chemokines (both C-C and C-X-C families) are also highly up-regulated, particularly MCP-1, 3, and 5, MIP-1α, Rantes, IP-10 and MIG. Chemokine CCL-5 has been previously detected in migratory T cells in the CNS of mice infected with RV (Galelli et al., 2000. J Neurovirol. 6:359-72). Recently, Nakamichi et al. (Nakamichi et al., 2004, J. Virol. 78:9376-88), reported that CXCL-10 was highly up-regulated and other chemokines were not up-regulated in RV-infected macrophages. The disparities between that study and ours reported here may be due to different types of cells involved. In the study by Nakamichi et al, (Nakamichi et al., 2004, J. Virol. 78:9376-88), only macrophages are used, whereas in the present study, the expression of chemokines is detected in the brain where there are other cell types beside neurons such as astrocytes, microglia and infiltrating CD3-positive T cells. It has been reported that chemokine (MCP-1) expression can also be affected by monocyte-astrocyte interactions (Andjelkovic et al., 2000. J Leukoc Biol. 68:545-52). Thus it is possible that interaction among neurons, astrocytes, microglia, and infiltrating CD3-positive T cells is responsible for the up-regulation of so many chemokines as observed in our present study. Activation of TLRs also induces inflammation (Boehme et al., 2004. J Virol. 78:7867-73). TLR1, TLR2, and TLR3 are all found to be up-regulated in B2C-infected mice. The increased expression of the chemokines, cytokines, and TLRs corresponds to the severe inflammatory reaction and significant increase in CD3-positive cell infiltration observed in mice infected with B2C. In addition, complement C1, particularly C1r, is highly up-regulated in B2C-infected mice. Although the classic or alternative complementary cascades may not be involved in RV resistance in the CNS (Hooper et al., 1998, J. Virol. 72:3711-9), increased expression of C1 may be a consequence of activated microglia during RV-induced CNS inflammation (Dietzschold et al., 1995, J. Neurol. Sci. 130:11-16). Inflammatory reaction and infiltration of T cells have been reported to play a major role in blocking RV spreading in the CNS (Baloul et al., 2003, Biochimie 85:777-88; Camelo et al., 2001. J. Virol. 75:3427-34), as well as RV clearance from the CNS (Hooper et al., 1998, J. Virol. 72:3711-9).

It is thus clear that attenuated RV activates the innate immune responses including the IFN-α/β pathway and inflammatory reactions. Up-regulation of these genes is detected in both IC- and IM-infected mice by both microarray and real-time PCR. In addition, infection of mice with other laboratory-adapted and attenuated RVs also resulted in up-regulation of genes involved in the innate immune responses. Furthermore, our data are supported by the companion paper that demonstrates that many of these genes involved in IFN signaling and inflammation are also up-regulated in human post-mitotic N2T cells after infection with laboratory-adapted RV (Préhaud et al., 2005. J. Virol. 79:12893-12904) On the other hand, pathogenic SHBRV induces very little or no inflammation and little or no up-regulation of gene expression in the IFN-α/β and inflammatory pathways. The activation of the innate immune responses by attenuated RV may play a protective role in the host against RV infection, which may explain why a few viral particles of the pathogenic RV can kill infected animals whereas about 1000 times more viral particles are required for the attenuated RV to kill infected animals in the mouse model. The evasion of the innate immune responses observed in SHBRV-infected mice may contribute to the highly neuroinvasive characteristic of the virus (Faber et al., 2004, Proc. Natl. Acad. Sci. USA, 101:16328-32; Morimoto et al., 1996, Proc. Natl. Acad. Sci. USA, 93:5653-8). IFN-α/β exerts its anti-viral activities by binding to IFN-α/β receptors, which activates STATs by phosphorylation (Stark et al., 1998, Amu Rev Biochem. 67:277-64). Phosphorylated STATs form specific multimeric complexes that translocate to the nucleus and initiate transcription (Darnell et al., 1994. Science. 264:1415-21). Stat1, Stat2, and Stat3 genes are all up-regulated in RV-infected mice as detected by the microarray hybridization and real-time PCR (see Tables 2 and 5). Furthermore, up-regulation of Stats expression resulted proportionally in increased protein synthesis. The level of STAT1 and STAT2 is higher in animals or cells infected with B2C than with SHBRV. On the other hand, STAT3 expression increases similarly in animal's or cells infected with either virus. . Most importantly, significantly more STAT1- and STAT2-translocated cells were found after infection with RV, particularly with B2C. Only a few STAT3-translocated cells were observed in RV infection. These data may indicate that RV infection, particularly with attenuated virus, not only results in the increased transcription and synthesis of STAT1 and STAT2, but also in the activation of the STAT 1 and STAT2, presumably by phosphorylation, leading to nuclear translocation. These data also suggest that STAT1 and STAT2, but not STAT3, are involved in the IFN-α/β activation and effector pathway in RV infections. This is in agreement with results from other studies that STAT1 and STAT2 promote the synthesis of effector proteins that inhibit viral replication (Aaronson et al., 2002. Science. 296:1653-5; Darnell et al., 1994. 264:1415-21). Increased expression of Stats has been reported in RV-infected mice (Préhaud et al., 2005. J. Virol. 79:12893-12904) and neuronal cells (Prosniak et al., 2001, Proc. Natl. Acad. Sci. USA, 98:2758-63).

To counter the host's anti-viral activities, viruses developed ways to impair the induction of innate immunity, particularly the IFN-α/β pathways (Samuel, 2001, Clin. Microbiol Rev. 14:778-809). Poxviruses encode soluble IFNR homologues that prevent IFN from acting through their natural receptors to elicit an anti-viral response (Smith et al., 1997, Immunol Rev. 159:137-54). Adenovirus VAI RNA antagonizes the anti-viral state of IFN by preventing PKR activation (Matthews et al., 1991, J. Virol. 65:5657-62). Poliovirus infection leads to the degradation of PKR (Black et al., 1993, J. Virol. 67:791-800). In a recent review, Conzelmann (Conzelmann. 2005. J. Virol. 79:5241-5248), summarized the mechanisms by which nonsegmented negative-stranded RNA viruses interfere with the transcriptional activation of IFN-α/β. For example, the V or the C protein from paramyxoviruses (Simian virus 5, Sendai virus, and mumps virus) mediates the degradation of STAT1 via ubiquitination pathway (Didcock et al., 1999. J Virol. 73:9928-33; Ohno et al., 2004, J. Gen. Virol. 85:2991-2999; Palosaari et al., 2003, J. Virol. 77:7635-7644; Shaffer et al., 2003, Virology, 315:389-397). The VP35 protein of Ebola virus inhibits the induction of IFN-β promoter and dsRNA/virus-mediated activation of ISRE-derived gene expression (Basler et al., 2000, Proc. Natl. Acad. Sci. USA, 97:12289-94). The NS1 protein of influenza virus is an INF-α/β antagonist (Garcia-Sastre et al., 1998, Virology, 252:324-330). The P protein of RV has recently been reported to interfere with the phosphorylation of IRF-3, thus exerting antagonistic function for IFN-α/β (Brzozka et al., 2005, J. Virol., in press). In our study, we found that the activation of the innate immune responses, particularly the up-regulation of IFN-α/β, correlates with the level of RV G expression. Not only in the CNS, but also in primary neurons, attenuated RV expresses abundantly the G while pathogenic RV expresses three-fold less G despite the fact that both viruses express similar amount of the N. The restriction of G expression also results in virus yield 3 logs lower in the brain of mice infected with wt SHBRV than those infected with B2C, similar to the findings by Faber et al. (Faber et al., 2004, Proc. Natl. Acad. Sci. USA 101:16328-32). Thus, we propose that one way by which pathogenic RV evades the innate immune responses is by restriction of the G expression. It has been reported that RV inhibits G expression in order to be pathogenic (Faber et al., 2002, J. Virol. 76:3374-81; Faber et al., 2004, Proc. Natl. Acad. Sci. USA, 101:16328-32; Morimoto et al., 2001, Vaccine, 19:3543-51). Thus it is possible that restriction of the G expression helps pathogenic RV to evade the innate immune responses. Although double-stranded RNA has been reported as the major factor for the induction of IFN-α/β in attenuated RV-infected cells (Préhaud et al., 2005. J. Virol. 79:12893-12904), it is also possible that RV G can activate the TLRs, particularly TLR-3, thus stimulating the expression of INF-α/β pathway in RV-infected cells. It has been reported that viral surface glycoproteins can activated TLRs (Boehme et al., 2004. J Virol. 78:7867-73). TLR-3 can sense RV infection in human post mitotic neurons to produce IFN-β (Préhaud et al., 2005. J. Virol. 79:12893-12904), and TLR-3 is also up-regulated in mice infected with RV in our studies.

In addition to genes involved in the innate immune and anti-viral responses, many other host genes such as those involved in apoptosis are also up-regulated in B2C, but not in SHBRV-infected mice. Up-regulation of more genes involved in apoptosis in B2C than SHBRV may explain the observation that B2C induced apoptosis (Morimoto et al., 1998, Proc. Natl. Acad. Sci. USA, 95:3152-3156), while SHBRV did not (Yan et al., 2001, J. Neurovirol 7:518-527). On the other hand, RV infection resulted in down-regulation of many of the neuron-specific genes. Actually there are more neuronal genes down-regulated than up-regulated, particularly in B2C-infected mice. This is not surprising since we have previously reported the down-regulation of neuron-specific genes such as the preproenkephlin gene by in situ hybridization in rats infected with CVS-24 (Fu et al., 1993, J. Virol. 67:6674-6681).

Furthermore, Prosniak et al. (Proc Natl Acad Sci USA. 98:2758-63, 2001) have reported that by using subtraction hybridization most the host genes were down-regulated in RV-infected mice. It is also found in the present study that there are as many transcriptional factors up-regulated as down-regulated in RV-infected mice. Previously we have reported that transcriptional factors such as egr-1 and c-jun are up-regulated in RV-infected rats (Fu et al., 1993, J. Virol. 67:6674-6681). The importance of the modification in the expression pattern for neuron-specific genes and the transcription factors in RV pathogenesis is not entirely clear and warrants further investigation.

TABLE 1
Host gene expression profiling in mice infected IC or IM with
B2C or SHBRV

| Number | B2C | SHBRV | Number | B2C | SHBRV |
|---|---|---|---|---|---|
| of genes | (IC) | (IC) | of genes | (IM) | (IM) |
| 419 | ▴ | ▴ | 215 | ▴ | ▴ |
| 62 | ▾ | ▾ | 63 | ▾ | ▾ |
| 373 | ▴ | | 671 | ▴ | |
| 237 | ▾ | | 631 | ▾ | |
| 0 | ▴ | ▾ | 4 | ▴ | ▾ |
| 2 | ▾ | ▴ | 0 | ▾ | ▴ |
| 104 | | ▴ | 44 | | ▴ |
| 45 | | ▾ | 131 | | ▾ |
| All other | | | All other genes | | |
| genes | | | | | |

▴: up-regulation,
▾: down-regulation,
: no up- or down-regulation

TABLE 2
Expression profile of genes involved in the IFN-α/β
signaling pathway in mouse brain infected with B2C or
SHBRV by IC or IM (fold change over the control mice).

| | Identification | | | |
|---|---|---|---|---|
| | B2C | SHBRV | B2C | SHBRV |
| Gene Category | (IC) | (IC) | (IM) | (IM) |
| Interferon genes | | | | |
| IFN-α2 | 2 | N | 3 | N |
| IFN-α4 | 8 | 6 | 9 | N |
| IFN-α5 | 9 | N | 8 | N |
| IFN-β | 3 | N | 3 | 2 |
| Interferon signaling genes | | | | |
| Cbp/p300-transactivator | 2 | 2 | 2 | N |
| Stat 1 | 32 | 18 | 14 | 9 |
| Stat 2 | 13 | 4 | 10 | 4 |
| Stat 3 | 4 | 5 | 5 | N |
| IRF -1 | 6 | 3 | 9 | 2 |
| IRF -2 | 3 | N | 5 | N |
| IRF -7 | 11 | 6 | 30 | 7 |
| Jak 2 | 2 | N | 2 | N |
| PKR | 3 | N | 6 | 4 |
| Interferon effector genes | | | | |
| 2′-5′ OAS-1B | 15 | 10 | 13 | 6 |
| 2′-5′ OAS-1G | 13 | 5 | 13 | 5 |
| 2′-5′ OAS-2 | 4 | N | 6 | 3 |
| 2′-5′ OAS-3 | 2 | N | 5 | N |
| 2′-5′ OAS -like 1 | 7 | 3 | 11 | 3 |
| 2′-5′ OAS -like 2 | 42 | 18 | 45 | 26 |
| ADAR, RNA-specific | 3 | 2 | −2 | N |
| MHC-I antigen | 3 | N | 4 | N |
| Mx 1 | 388 | 91 | 119 | 18 |
| Mx 2 | 56 | 15 | 147 | 24 |
| Interferon activated genes | | | | |
| IFN activated gene 202B | 315 | 147 | 478 | 49 |
| IFN activated gene 203 | 5 | 2 | 23 | N |
| IFN activated gene 204 | 49 | 12 | 478 | 16 |
| IFN activated gene 205 | 45 | 11 | 147 | 12 |
| IFN -induced protein 1* | 37 | 21 | 24 | N |
| IFN -induced protein 2* | 256 | 128 | 208 | 74 |
| IFN -induced protein 3* | 45 | 18 | 60 | 30 |

*IFN -induced transmembrane protein with tetratricopeptide repeats 1, 2, and 3.
N: no change.

TABLE 3
Expression profile of inflammatory genes in mouse
brain infected with B2C or SHBRV by IC or IM routes
(fold change over the control mice).

| | Identification | | | |
|---|---|---|---|---|
| | B2C | SHBRV | B2C | SHBRV |
| Gene Category | (IC) | (IC) | (IM) | (IM) |
| Toll-like receptors | | | | |
| TLR1 | 3 | N | 2 | 2 |
| TLR2 | 6 | 4 | 5 | N |
| TLR3 | 10 | 6 | 11 | 4 |
| Chemokines | | | | |
| chemokine (C-C) MCP-5 | 60 | 60 | 39 | 37 |
| chemokine (C-C) MCP-1 | 28 | 8 | 12 | 3 |
| chemokine (C-C) MIP-1α | 104 | 34 | 111 | N |
| chemokine (C-C) MIP-1 β | 5 | N | 4 | N |
| chemokine (C-C) RANTES | 56 | 12 | 91 | 5 |
| chemokine (C-C) MCP-3 | 37 | 11 | 7 | 3 |
| chemokine (C-X-C) MIP-2 α | 6 | 7 | 3 | 3 |
| chemokine (C-X-C) IP-10 | 84 | 37 | 158 | 26 |
| chemokine (C-X-C) L11 | 16 | 3 | 25 | 3 |
| chemokine (C-X-C) BLC | 3 | N | 3 | N |
| chemokine (C-X-C) MIP-2 β | 4 | 5 | 3 | 3 |
| chemokine (C-X-C) MIG | 34 | 5 | 42 | 3 |
| Cytokines | | | | |
| IL-6 β | 4 | 3 | 15 | 3 |
| IL-12 | 3 | 2 | N | N |
| IL-12 receptor, beta 1 | 3 | N | 20 | 11 |
| IL-13 receptor, alpha 1 | 5 | N | N | N |
| TGF-β regulated gene 1 | 3 | N | 2 | N |
| TGF-β induced, 68 kDa | 7 | 2 | 28 | N |
| TNFR-12α | 15 | 17 | 7 | N |
| TNFR-5 | 2 | N | N | N |
| TNFα-induced protein 2 | 7 | 8 | 3 | N |
| Complement genes | | | | |
| complement c1q- α | 2 | 2 | N | N |
| complement c1q- β | 3 | 3 | 2 | 2 |
| omplement c1q- γ | 3 | 2 | 3 | N |
| complement c1r | 56 | 20 | 18 | 2 |
| complement c1s | 3 | N | 158 | 5 |
| complement c2 | 4 | N | 4 | 2 |
| complement c3 | 30 | 8 | 119 | N |
| complement c4 | 5 | 4 | 8 | 4 |

N: no change.
TGF, transforming growth factor; TNF, tumor necrosis factor; TNFR, TNF receptor.

TABLE 4
Primers used for real-time-PCR.
(SEQ ID NOs. 9-34)

| | Forward primer | Reverse primer |
|---|---|---|
| Genes | (5′ . . . 3′) | (5′ . . . 3′) |
| GAPDH | GGAGAAGCTGCCAATGGATA | TTACGCTTGCACTTCTGGTG |
| IFNα-2 | TCTGTGCTTTCCTCGTGATG | TTGAGCCTTCTGGATCTGCT |
| IFNα-5 | CTGCCTGAAGGACAGAAAGG | TCATTGAGCTGCTGATGGAC |
| Stat1 | CACATTCACATGGGTGGAAC | TCTGGTGCTTCCTTTGGTCT |
| Stat2 | ACCAGTGGGACCACTACAGC | ATCTCAAGCTGCTGGCTCTC |
| Stat3 | TCATGGGTTTCATCAGCAAG | TGGTCGCATCCATGATCTTA |
| IRF2 | CTTATCCGAACGACCTTCCA | CTTGCTGTCCAGATGGGACT |
| IRF7 | CCTCTTGCTTCAGGTTCTGC | GGCCCTTGTACATGATGGTC |
| OAS-1G | GGCTGTGGTACCCATGTTTT | CAGAAGCACGGAATCTGATG |
| Mx1 | CAAAGCCCTGGAAGAGTCTG | CGGATCAGGTTTTCAGCTTC |
| MCP-1 | AGGTCCCTGTCATGCTTCTG | TCTGGACCCATTCCTTCTTG |
| IP-10 | GGTCTGAGTGGGACTCAAGG | TCTTTTTCATCGTGGCAATG |
| Rantes | CCCTCACCATCATCCTCACT | CCTTCGAGTGACAAACACGA |

TABLE 5
Comparison between the real-time-PCR results and the Microarray
data in fold increase in mice infected with either SHBRV or
B2C over the controls.

| | Microarray data | | | Real-time-PCR results | | |
|---|---|---|---|---|---|---|
| | B2C/ | SHBRV/ | B2C/ | B2C/ | SHBRV/ | B2C/ |
| Genes | Control | Control | SHBRV | Control | Control | SHBRV |
| IFN-α2 | 2 | NC | NA | 2 | 1.3 | 1.6 |
| IFN-α5 | 9 | NC | NA | 3.1 | 1.9 | 1.6 |
| Stat1 | 32 | 18 | 1.8 | 55 | 22 | 2.5 |
| Stat2 | 13 | 4 | 3.3 | 19 | 4.8 | 4.4 |
| Stat3 | 4 | 5 | 0.8 | 6 | 3.3 | 1.8 |
| IRF2 | 3 | NC | NA | 2.3 | 1.1 | 2 |
| IRF7 | 11 | 6 | 1.9 | 25 | 12 | 2 |
| OAS-1G | 13 | 5 | 2.6 | 78.6 | 37 | 2.1 |
| Mx1 | 388 | 91 | 4.3 | +∞* | +∞ | 6 |
| MCP-1 | 28 | 8 | 3.5 | 11 | 4.6 | 2.4 |
| IP-10 | 84 | 37 | 2.3 | 2394 | 819 | 2.9 |
| Rantes | 56 | 12 | 4.8 | 413 | 267 | 1.5 |

NC: no change when compared with control;
NA: not applicable;
*no signal for control.

### Example III

### Comparison of SHBRV, B2C and L16 Rabies Virus

Profile of Host Gene Expression

In Example II we profiled host gene expression in a mouse model using two viruses, SHBRV and B2C. Here we describe a further comparison with L16. L16 is cloned from the vaccine strain, SAD-B19, by reverse genetics technology (Schnell et al. Infectious RVes from cloned cDNA. EMBO J. 1994; 13:4195-4203; Wu et al. “Both viral transcription and replication are reduced when the rabies virus nucleoprotein is not phosphorylated.” J. Virol. 2002; 76:4153-61). Initially we compared the virulence of these viruses. Virus titers were determined in BHK cells and measured as focus forming unit (ffu) and the 50% intracerebral lethal dose (ICLD₅₀) and the 50% intramuscular lethal dose (IMLD₅₀) were determined in mice. The IC and IM pathogenic indices were calculated by subtracting the log virus titer/nil in BHK cells from the log ICLD₅₀/ml or the log IMLD₅₀/ml and the results are shown in Table 6. 1000 to 10,000 times more viral particles are required for attenuated B2C and L16 than for SHBRV to kill infected mice by either the IC or IM routes, suggesting that SHBRV is more virulent than attenuated B2C and L16 in the mouse model.

TABLE 6
Pathogenic indices of three RV by
either IC or IM route of infection

| Viruses | IC pathogenic index | IM pathogenic index |
|---|---|---|
| B2C | 1.1 × 10⁴ | 6.2 × 10⁷ |
| L16 | 6.6 × 10⁴ | ND |
| SHBRV | 0.15 | 3.3 × 10⁴ |

TABLE 7
Expression profile of genes involved in the IFN-β signaling
pathway in mouse brain infected with attenuated (B2C and L16) or
virulent (SHBRV) by IC or IM (fold change over the control mice).

| | B2C | L16 | SHBRV | B2C | SHBRV |
|---|---|---|---|---|---|
| Gene Category/Identification | (IC) | (IC) | (IC) | (IM) | (IM) |
| Interferon genes | | | | | |
| IFN-α2 | 2 | N | N | 3 | N |
| IFN-α4 | 8 | 5 | 6 | 9 | N |
| IFN-α5 | 9 | 3 | N | 8 | N |
| IFN-β | 3 | N | N | 3 | 2 |
| Interferon signaling genes | | | | | |
| Cbp/p300-transactivator | 2 | 2 | 2 | 2 | N |
| Stat 1 | 32 | 30 | 18 | 14 | 9 |
| Stat 2 | 13 | 11 | 4 | 10 | 4 |
| Stat 3 | 4 | 6 | 5 | 5 | N |
| IRF-1 | 6 | 8 | 3 | 9 | 2 |
| IRF-2 | 3 | 2 | N | 5 | N |
| IRF-7 | 11 | 11 | 6 | 30 | 7 |
| Jak 2 | 2 | 2 | N | 2 | N |
| PKR | 3 | 2 | N | 6 | 4 |
| Interferon effector genes | | | | | |
| 2′-5′ OAS-1B | 15 | 10 | 10 | 13 | 6 |
| 2′-5′ OAS-1G | 13 | 13 | 5 | 13 | 5 |
| 2′-5′ OAS-2 | 4 | 5 | N | 6 | 3 |
| 2′-5′ OAS-3 | 2 | 2 | N | 5 | N |
| 2′-5′ OAS-like 1 | 7 | 5 | 3 | 11 | 3 |
| 2′-5′ OAS-like 2 | 42 | 34 | 18 | 45 | 26 |
| ADAR, RNA-specific | 3 | 4 | 2 | −2 | N |
| MHC-I antigen | 3 | 3 | N | 4 | N |
| Mx 1 | 388 | 294 | 91 | 119 | 18 |
| Mx 2 | 56 | 32 | 15 | 147 | 24 |
| Interferon activated genes | | | | | |
| IFN activated gene 202B | 315 | 338 | 147 | 478 | 49 |
| IFN activated gene 203 | 5 | 4 | 2 | 23 | N |
| IFN activated gene 204 | 49 | 34 | 12 | 478 | 16 |
| IFN activated gene 205 | 45 | 64 | 11 | 147 | 12 |
| IFN-induced protein 1* | 37 | 37 | 21 | 24 | N |
| IFN-induced protein 2* | 256 | 275 | 128 | 208 | 74 |
| IFN-induced protein 3* | 45 | 45 | 18 | 60 | 30 |

*IFN-induced transmembrane protein with tetratricopeptide repeats 1, 2, and 3.
N: no change.

To profile the host gene expression, mice were infected with 10 ICLD₅₀ of SHBRV, B2C, and L16 by the IC route, as described in Example II. Mice were also infected with 10 IMLD₅₀ of SHBRV and B2C by the IM route, as described in Example II. Mice were sacrificed when showing paralysis and brains were harvested by flash freezing. Sham-infected mice were used as controls. Total RNA was extracted from the brain and used for cRNA synthesis. The cRNA was then used to hybridize to the mouse expression set 430A genomic Array (Affymetrix). The data were analyzed by a combination of GeneChip Operating Software (Affymetrix) and dChip method (Harvard University). The normalized data for 22,626 mouse genes were collected. Changes over two-fold are considered for either up- or down-regulation. Analysis of the microarray data by gene ontology revealed that virulent and attenuated RVs differentially induce host gene expression, particularly the innate immune and antiviral genes. Attenuated B2C and L16 induced the expression of genes in the IFN-β pathway and inflammatory chemokines. On the other hand, wt SHBRV is a poor inducer of the innate immune responses (Table 7) (Wang et al., 2005, J. Virol. 79:12554-12565; Example II). In mice infected with B2C and L16 by the IC or IM routes, most of the genes involved in IFN-β pathway are up-regulated. These include IFN-β genes, genes involved in IFN-mediated signaling and transcription activation, and genes encoding proteins implicated in the anti-viral activities. Up-regulated IFN genes include IFN-α2, α4, and α5 as well as β. Interferon signaling genes (Cbp/p300, Stat1, 2, 3, and Jak-2) and interferon regulatory factors (IRF-1, 2, and 7) are up-regulated. IFN-β induced proteins implicated in anti-viral activities, including double-stranded RNA-dependent protein kinase (PKR), RNA-specific adenosine deaminase (ADAR), the 2′,5′-oligoadenylate synthetases (OAS), myxovirus resistance (Mx), and MHC class I are also up-regulated in B2C and L16-infected animals. The up-regulated genes for 2′5′-OAS include 1B, 1G, 2, and 3 as well as OAS-like 1 and 2. Along the IFN signaling pathway, many IFN-activated or inducible genes (IFN activated gene 202B, 203, 204, and 205, IFN-induced transmembrane protein with tetratricopeptide repeats 1, 2, and 3) are highly up-regulated. The mostly up-regulated gene is the anti-viral Mx1 that is increased by 388 fold in mice infected with B2C by the IC route. The magnitude of activation of the gene involved in IFN-α/β signaling is similar in mice infected with B2C and L16.

On the other hand, many of genes important in the IFN-α/β pathway are not up-regulated in SHBRV-infected mice. The IFN genes are not up-regulated except IFN-α4 (6 fold) by IC and IFN-β (2 fold) by IM. For the IFN signaling and effetor genes, Cbp/p300 transactivator, Stat3, Jak-2, IRF-2, 2′5′-OAS-2, -3, ADAR, MHC-1, PKR, IFN activated gene 203, and IFN-induced transmembrane protein with tetratricopeptide repeats 3 are not up-regulated in mice infected with SHBRV by the IC or IM routes. Some of the genes in the IFN pathway are up-regulated in mice infected with SHBRV but the increase was 2 to 30-fold lower than with B2C or L16 (Table 7).

TABLE 8
Expression profile of inflammatory chemokine genes in mouse
brain infected with attenuated B2C and L16 or virulent SHBRV
by IC or IM routes (fold change over the control mice).

| | B2C | L16 | SHBRV | B2C | SHBRV |
|---|---|---|---|---|---|
| Gene Category/Identification | (IC) | (IC) | (IC) | (IM) | (IM) |
| Toll-like receptors | | | | | |
| TLR1 | 3 | 3 | N | 2 | 2 |
| TLR2 | 6 | 5 | 4 | 5 | N |
| TLR3 | 10 | 6 | 6 | 11 | 4 |
| Chemokines | | | | | |
| chemokine (CCL2) MCP-1 | 28 | 23 | 8 | 12 | 3 |
| chemokine (CCL3) MIP-1α | 104 | 169 | 34 | 111 | N |
| chemokine (CCL4) MIP-1β | 5 | 9 | N | 4 | N |
| chemokine (CCL5) RANTES | 56 | 45 | 12 | 91 | 5 |
| chemokine (CCL7) MCP-3 | 37 | 30 | 11 | 7 | 3 |
| chemokine (CCL12) MCP-5 | 60 | 45 | 60 | 39 | 37 |
| chemokine (CXCL1) MIP-2α | 6 | 3 | 7 | 3 | 3 |
| chemokine (CXCL2) MIP-2β | 4 | 2 | 5 | 3 | 3 |
| chemokine (CXCL9) MIG | 34 | 39 | 5 | 2 | 3 |
| chemokine (CXCL10) IP-10 | 84 | 79 | 37 | 58 | 26 |
| chemokine (CXCL11) | 16 | 21 | 3 | 25 | 3 |

Components in the inflammatory pathway including toll-like receptors (TLR) and chemokines are also up-regulated in B2C and L16-infected animals (Table 8). The expression of TLR1, TLR2, and TLR3 is up-regulated. Pro-inflammatory chemokines in both the C═C and C═X═C families including Rantes (CCL5), MCP-1 (CCL2), MCP-3 (CCL7), MCP-5 (CCL12), MIP-1α(CCL3.), MIP-1β(CCL4), MIP-2α(CXCL-1), MIP-2β(CXCL-2.), and IP-10 (CXCL-10) are all up-regulated with some increased more than 100 folds. The magnitude of activation of the TLR and chemokine genes is similar in mice infected with B2C or L16. In mice infected with SHBRV, TLR1 and TLR2 are not up-regulated. For chemokines, only MCP-5 is up-regulated in SHBRV-infected mice in a similar level as in B2C or L16-infected animals. MIP-1α,1α and CXCL 11 are not up-regulated in mice infected with SHBRV by IC or IM. The up-regulation of other chemokines in mice infected with SHBRV is 2 to 20-fold lower than that in mice infected with B2C or L16 (Table 8).

The gene profiling data indicate that attenuated RV (B2C and L16) are potent activators of the innate immune responses including the IFN-β induction and signaling pathway and inflammatory pathway. In contrast, SHBRV evades the innate immune responses, thus contributing to its virulence, which could explain why a few viral particles of the virulent RV (SHBRV) can kill infected animals while 1000 to 10000 more viral particles are required by attenuated RV (B2C and L16) to kill infected animals (Table 6). Thus, induction of innate immune responses is am important mechanism for RV attenuation.

### IFN-α Inhibits RV Replication

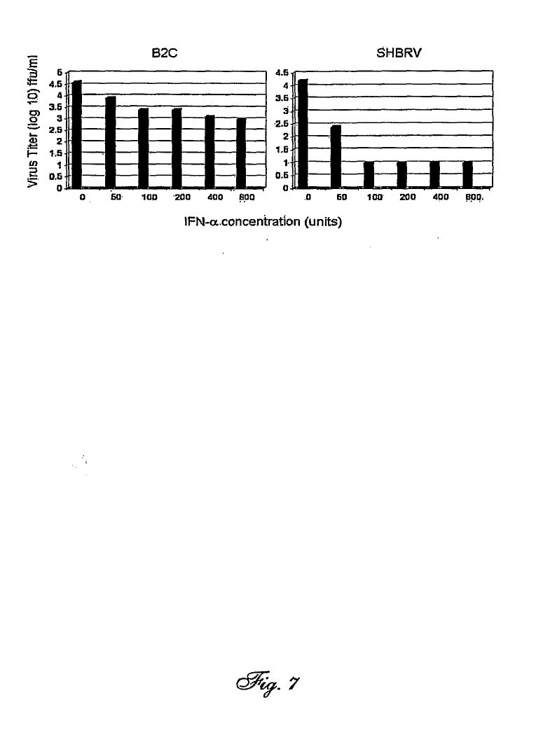

Up-regulation of IFN-α/β induction and signaling pathway could have direct anti-viral effects. To determine if IFN inhibits RV replication, NA cells were treated with IFN-α at concentrations of 50, 100, 200, 400, and 800 units. Twenty-four hrs later, the cells were infected with B2C or SHBRV at 0.1 ffu/cell. After further incubation for 48 hrs, the supernatants were harvested for virus titration. As shown in FIG. 7, treatment with 50 units of IFN resulted in 2 logs reduction in virus production of SHBRV and SHBRV replication was almost completely inhibited by treatment with 100 units of IFN. Treatment with 800 units of IFN resulted in 2 logs reduction of B2C. These data indicate that virulent RV is more sensitive to IFN than attenuated RV, which may explain why virulent RV evolved ways to evade the host innate immune responses.

Attenuated RV Induced More Inflammation than Virulent RV

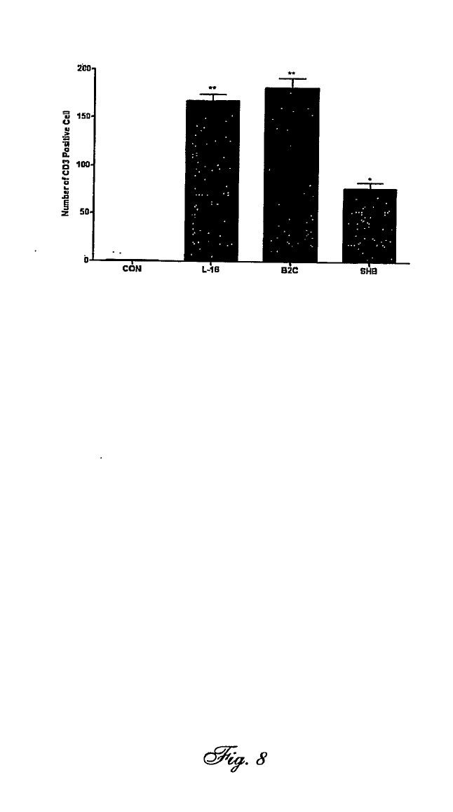

Inflammatory chemokines can recruit neutrophils, monocytes, and lymphocytes. To examine if infection with attenuated RVs results in more inflammation in the mouse brain than with virulent RV, mice were infected with B2C, L16, or SHBRV and transcardially perfused when showing paralysis. Brains were removed for histology and immunohistochemistry. It was found that attenuated B2C and L16 induced extensive pathological changes, particularly inflammation including perivascular cuffing, gliosis, and infiltration of macrophages and lymphocytes. On the other hand, only a few pathological changes were observed in mice infected with SHBRV. To quantify the inflammatory reactions, CD3-positive T cells were measured using anti-CD3 antibodies in the cortex. Three serial sections were selected from each mouse for enumeration and the average number of CD3-positive cells was obtained and analyzed statistically by one way ANOVA. As shown in FIG. 8 significantly (p<0.01) more CD3-positive T cells were detected in B2C- and L16- than in SHBRV-infected mice, indicating that more inflammatory cells infiltrated attenuated RV-infected than virulent RV-infected mouse brain (Wang et al., 2005, J. Virol. 79:12554-12565; 75; Sarmento et al., 2005, J. Neurovirol. 11:571-581; Example IV.

### Example IV

### Glycoprotein-Mediated Induction of Apoptosis Limits the Spread of Attenuated Rabies Viruses in the CNS of Mice

Induction of apoptosis by rabies virus (RV) has been reported to be associated with the expression of the glycoprotein (G), but inversely correlated with pathogenicity. To further delineate the association between the expression of the G and the induction of apoptosis, recombinant RVs with replacement of only the G gene were used to infect mice by the intracerebral route. Recombinant viruses expressing the G from attenuated viruses expressed higher level of the G and induced more apoptosis in mice than recombinant RV expressing the G from wild-type (wt) or pathogenic RV, demonstrating that it is the G gene that determines the level of G expression and, consequently, the induction of apoptosis. Likewise, recombinant viruses expressing the G from wild-type (wt) or pathogenic RV are more pathogenic in mice than those expressing G from attenuated RV, confirming the inverse correlation between RV pathogenicity and the induction of apoptosis. To investigate the mechanism by which induction of apoptosis attenuates viral pathogenicity, mice were infected with wt or attenuated RV by the intramuscular route. It was found that low doses of attenuated RV induced apoptosis in the spinal cord and failed to spread to the brain or produce neurological disease. On the other hand, apoptosis was not observed in the spinal cord of mice infected with the same doses of wt RV and the virus spread to various parts of the brain and induced fatal neurologic disease. These results suggest that glycoprotein-mediated induction of apoptosis limits the spread of attenuated rabies viruses in the CNS of mice.

### Background

Apoptosis, or programmed cell death, is the process whereby individual cells of multicellular organisms undergo systematic self-destruction in response to a wide variety of stimuli and the main characteristics are cellular shrinkage, membrane condensation, membrane blebbing, and DNA fragmentation. Apoptosis plays an important physiological role in normal embryonic development and tissue homeostasis and is also a common response of cells to virus infections.

Virus-induced apoptosis has been suggested both as pathological and protective responses of the host (Mori et al, 2004. Rev Med Virol. 14: 209-216). In some circumstances, virus-induced apoptosis can contribute to pathogenesis, for example, destruction of many cells by apoptosis, particularly those nonreplenishable cells such as neurons, may result in diseases (Lewis et al, 1996. J Virol 70: 1828-1835). Indeed, the ability to induce apoptosis in neurons has been correlated with neurovirulence for alphavirus and flavivirus (Lewis et al, 1996. J Virol 70: 1828-1835; Despres et al, 1998. J Virol 72: 823-829). More typically, however, apoptosis represents an important host defense mechanism (Barber et al., 2001. 8:113-126; Kerr et al., 1991. Cold Spring Harbor, N.Y.: Cold Spring Harbor Laboratory Press). Cells evolved to commit suicide upon viral infection to exterminate unwanted intracellular pathogens from tissues, organs or a whole organism (Mori et al, 2004. Rev Med Virol. 14: 209-216). In addition, death by apoptosis instead of necrosis can significantly affect the efficiency of viral antigen capture by antigen-presenting cells and presentation to T cells, thus enhancing adaptive immune responses as well (Barber et al., 2001. 8:113-126).

Both beneficial and detrimental effects of apoptosis have been suggested in RV infections. In experimental animals infected intracerebrally (IC) with mouse-adapted CVS virus, extensive apoptosis was observed in the CNS (Jackson et al., 1997. J Virol 71: 5603-5607; Theerasurakarn et al., 1998. J Neurovirol 4: 407-414). These observations led to the hypothesis that apoptosis plays an important pathogenic role in experimental RV infections. However, Morimoto et al., 1999. J Virol 73: 510-518, found that the ability of a RV to induce apoptosis in primary neuronal cultures correlated inversely with its pathogenicity in animals. In addition, extensive apoptosis was observed in mice infected with laboratory-adapted CVS-24, but not in mice infected with a street RV strain, SHBRV-18 (Yan et al., 2001. J Neurovirol 7: 518-527). Recently, Thoulouze et al. (2003) Ann N Y Acad Sci. 1010:598-603), observed an inverse correlation between the induction of apoptosis and the capacity of a RV strain to invade the brain, suggesting that inhibition of apoptosis could be a strategy employed by neurotropic virus to favor its progression through the nervous system. Thus, induction of apoptosis is a host defense mechanism in RV infections. This hypothesis is further supported by the findings that recombinant RV expressing cytochrome c induced more apoptosis than parental virus and attenuated its pathogenicity (Pulmanausahakul et al., 2001. J Virol. 75:10800-7).

It has also been reported that induction of apoptosis correlates with the level of G expression (Morimoto et al., 1999. Proc Natl Acad Sci USA. 93:5653-8); Yan et al., 2001. J Neurovirol 7: 518-527; Faber et al., 2002. J Neurovirol 7: 518-527). In the present study, different recombinant RVs that differ only in the G gene were used to infect mice and the level of G expression was correlated with the induction of apoptosis in the brain. It was found that recombinant viruses expressing the G from attenuated viruses expressed higher levels of the G and induced more apoptosis than those expressing wt or pathogenic RV G, demonstrating that G gene determines the level of G expression, and consequently, the induction of apoptosis. Furthermore, attenuated RV induced apoptosis in the spinal cord and failed to spread to the brain, whereas little to no apoptosis was detected in the spinal cord of mice infected with wt RV and the virus spread to various regions of the brain, suggesting that G-mediated induction of apoptosis limits the spread of attenuated rabies viruses in the CNS of mice.

### Materials and Methods

### Viruses, Cells, and Antibodies

Seven different RV strains were used in this study including four parental viruses (SN-10, B2C, N2C and SHBRV) and three recombinant viruses (RB2C, RN2C and RSHBRV). All these viruses were obtained from Dr. Bernhard Dietzschold, Thomas Jefferson University. Virus stocks were prepared as described (Morimoto et al., 1996. Proc Natl Acad Sci USA. 93:5653-8; Schnell et al., 1994. EMBO J. 13:4195-203; Yan et al., 2002. J Neurovirol. 8:345-52). Briefly, one-day-old suckling mice were infected with 10 μl of viral samples by the IC route. When moribund, mice were sacrificed and brains removed. A 20% (w/v) suspension was prepared by homogenizing the brain in Dulbecco's modified Eagle's medium (DMEM). The homogenate was centrifuged to remove debris and the supernatant collected and stored at −80° C. Baby hamster kidney (BHK) cells were cultured in DMEM. Anti-RV N monoclonal antibody 802-2 (Hamir et al., 1995) was obtained from Dr. Charles Rupprecht, Center for Disease Control and Prevention. Anti-RV G polyclonal antibody was prepared in rabbit as described (Fu et al., 1993. Antisense Research and Development, 6:87-93).

### Mouse Primary Neuronal Cultures

Mouse primary neuronal cultures were prepared using standardized procedures as described (Adamec et al., 2001. Brain Res. Protocol 7:193-202; Li et al., 2005. J. Virol. 79:10063-10068; Wang et al., 2005. J. Virol. 79:12554-12565; Example II). Swiss-Webster mice at gestation day 16 were euthanized and the embryos removed. Neocortex from these embryos were collected and digested with trypsin, separated neuronal cells were plated into culture wells treated with poly-D-lysine (50 μg/ml). The primary neurons were grown in Neurobasal medium supplemented with 2% B-27, 500 mM glutamine, 25 mM glutamate, 10% fetal bovine serum, and 1% horse serum in a humidified atmosphere of 5% CO₂-95% air at 37° C. Ara-c (cytosine furo-arabinoside) at a final concentration of 1 μM was added at 1 and 5 days after plating to prevent the proliferation of non-neuronal cells.

Animal Infection and Tissue Collection

ICR mice (Harlan) at the age of 4-6 weeks were housed in temperature- and light-controlled quarters in the Animal Facility, College of Veterinary Medicine, University of Georgia. They had access to food and water ad libitum. Mice were infected with 10 ICLD₅₀ of each virus by the IC route. Alternatively, mice were infected with different doses of RVs by the IM route in the hind legs (both sides). Infected animals were observed twice daily for 20 days for the development of rabies. Sham-infected mice were included as controls. At the time of severe paralysis or at different time points after virus infection, mice were anesthetized with ketamine/xylazine at a dose of 0.2 ml and then perfused by intracardiac injection of PBS followed by 10% neutral buffered formalin as described (Yan et al., 2001. J Neurovirol 7: 518-527; Yan et al., 2002. J Neurovirol. 8:345-52). Only brains were removed from mice infected by IC while both spinal cords and brains were collected from the IM-infected mice. Tissues collected were placed in the same fixative (10% neutral buffered formalin) for one week at 4° C. Tissues were paraffin embedded and coronal sections (4 μm) were obtained and placed on glass slides.

### Determination of Virus Titers, LD₅₀, and Pathogenic Index

Virus titers were determined in BHK cells as described (Fu et al., 1996. Antisense Res Dev, 6:87-93). Briefly, virus preparations were serially (10-fold) diluted in 96-well plates and cell suspension was added into each well. After 24 hours of incubation at 37° C., infected cells were fixed in 80% acetone and viral antigen was detected with FITC-conjugated anti-RV antibodies (FujiRab, Malvin, Pa.). Infectious foci were counted under a fluorescence microscope and calculated as focus-forming units (ffu) per milliliter (ffu/ml). All titrations were performed in duplicate, and the average infectious foci were used to determine the virus titer. LD₅₀ of individual viruses was determined by infecting 4 to 6-week-old ICR mice by either the IC or the IM route as described (Morimoto et al., 2001. Vaccine. 19:3543-51). Essentially, virus was serially (10-fold) diluted in DMEM and 10 μl of each dilution was used to infect each mouse. For each virus dilution, 10 mice were used. Infected animals were observed twice daily for 20 days for the development of rabies (paralysis and death). IC or IM LD₅₀ were calculated as described by Reed et al., 1938. The American Journal of Hygine: 27(3) 493-497. RV pathogenic index was determined by the following formula: log ICLD50/ml divided by the log virus titer/ml as described (Morimoto et al., 1998. Proc Nat Acad Sci USA. 95: 3152-3156; Morimoto et al., 2001. Vaccine. 19:3543-51).

### Histopathology and Immunohistochemistry

Histopathology was performed by staining the paraffin embedded tissue sections with H&E or cresyl violet. Severity of the lesions was scored according to the degree of vacuolation, inflammation, and necrosis. Lesions were classified as ++++ (most severe), +++ (severe), ++ (moderate), + (mild), or − (none).

For immunohistochemistry, paraffin-embedded brain and spinal cord tissue sections were heated at 70° C. for 10 minutes, then dipped in Hemo-De for 3×5min and dried until chalky white. Slides were incubated with proteinase K (20 ug/ml in 10 mM tris.HCL pH 7.4-8.0) for 15 min at 37° C. and rinsed for 3×5 min with PBS. For detection of apoptosis, the TdT-mediated deoxyuridine triphosphate-biotin nick end labeling (TUNEL) assay was performed using the In situ Cell death Detection kit, AP (Roche Scientific) as described previously (Yan et al., 2001. J Neurovirol 7: 518-527). Briefly, TUNEL reaction mixture was added onto the slides covered with cover slips and incubated for 60 minutes at 37° C. Converter-AP was then added to each slide (approximately 1000 and incubated again for 30 min at 37° C. Slides were rinsed 3×5 min with PBS and substrate (NBT and BCIP) or Vulcan Fast Red (Biocare) was added. After color development, slides were counterstained with methyl green or hematoxylin and mounted with mounting media. TUNEL-positive cells were counted and analyzed statistically by one-way ANOVA and Student's t-Test.

For viral antigen detection, tissue slides were incubated with either the anti-RV N monoclonal antibody (802-2) (Hamir et al., 1995. Vet Rec 136: 295-296), or with the anti-RV G polyclonal antibody as described previously (Yan et al., 2001. J Neurovirol 7: 518-527). The secondary antibody used was biotinylated goat anti-mouse or goat anti-rabbit IgG from the VectaStain kits (Vectorlab). The avidin-biotin-peroxidase complex (ABC) then was used to localize the biotinylated antibody. Finally diaminobenzidine (DAB) was used as a substrate for color development. For double-labeling, spinal cord sections were detected first with anti-RV antibodies followed by TUNEL assay.

### Results

### Pathogenicity of Recombinant RVs is Largely Determined by the G

In this study, four parental viruses (SN-10, B2C, N2C and SHBRV) and three recombinant viruses (RB2C, RN2C and RSHBRV) were used to determine the association between RV G and the pathogenicity. Among the four parental viruses, SHBRV is a wt RV isolated from a human patient (Rupprecht et al., 1997. J Neurovirol. Suppl 1:S52-3; Morimoto et al., 1996. Proc Natl Acad Sci USA. 93:5653-8), and has been associated with most of the human rabies cases in the United States for the past 15 years. The other three viruses are laboratory-adapted viruses. N2C and B2C were isolated from CVS-24 by passaging in neuroblastoma and BHK cell lines, respectively (Morimoto et al., 1998. Proc Nat Acad Sci USA. 95: 3152-3156). SN-10 is a clone generated from the vaccine strain, SAD-B 19, by reverse genetics technology (Schnell et al., 1994. EMBO J. 13:4195-203). The three recombinant viruses (RB2C, RN2C and RSHBRV) were obtained by reverse genetics using a SN-10 viral genomic backbone, replacing the G gene with G genes from B2C, N2C, or SHBRV (Morimoto et al., 2001. Vaccine. 19:3543-51).

To determine the pathogenicity, virus titers and ICLD₅₀ of the different virus stocks were measured in BHK cells and mice (by IC route of infection), respectively. The pathogenic index for a particular virus is the log ICLD₅₀/ml divided by the log virus titer/ml in BHK cells (Morimoto et al., 1998, 2001). Virus titers, ICLD50, and the pathogenic index for each of these viruses are summarized in Table 9. Among the seven viruses tested, SHBRV and N2C were the most pathogenic viruses, followed in order by RSHBRV, RN2C, B2C, SN-10, and RB2C. Overall, recombinant viruses were found to be less pathogenic than the parental viruses. However, recombinant viruses expressing the G from pathogenic viruses such as SHBRV and N2C were more pathogenic than attenuated strains such as B2C and SN-10. These results indicate that the G is largely the determinant for viral pathogenicity.

More Apoptotic Cells were Observed in Mice Inoculated IC with Attenuated than Pathogenic Viruses

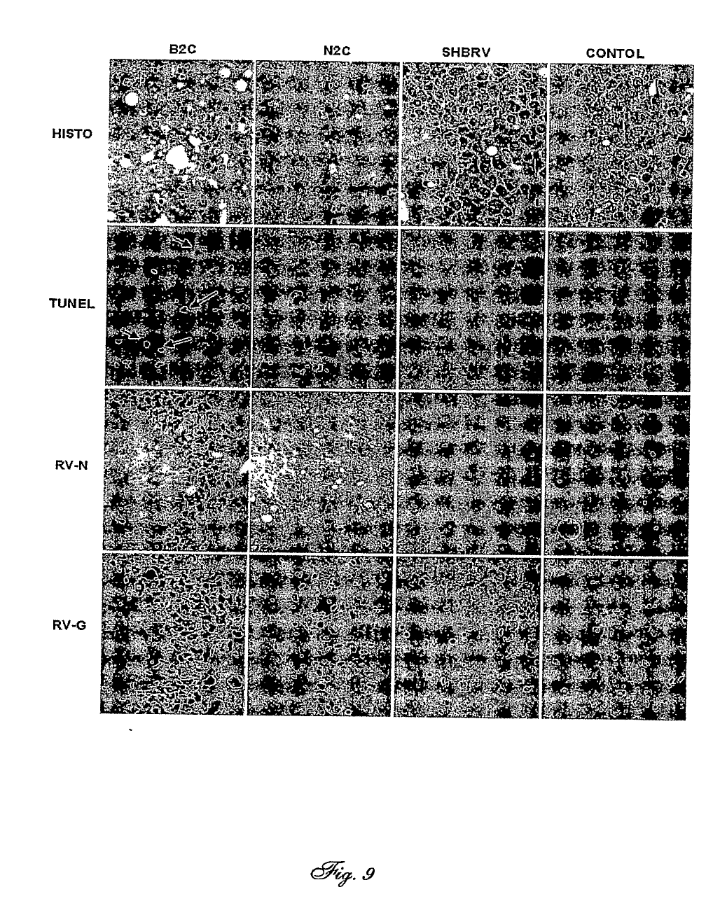

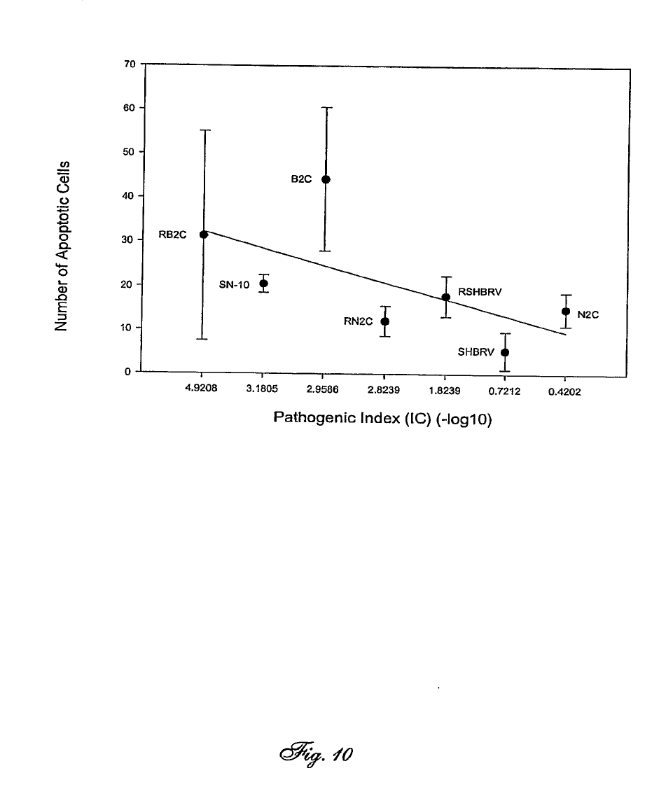

To examine histopathological lesions, mice were infected with 10 ICLD₅₀ of each virus and brains were harvested at the time when mice developed paralysis. Brain sections from 4 mice infected with each virus were stained with hematoxylin and eosin (H&E). Brain sections from sham-infected mice were also included. Overall, pathological changes included apoptosis, necrosis, inflammation, vacuolation, and gliosis (FIG. 9). The severity of histological lesions was scored for each virus and is summarized in Table 9. The most severe histopathological changes were observed in mice infected with B2C and RB2C, whereas SN-10, N2C, RN2C and RSHBRV infected mice had moderate histopathological changes. Mice infected with SHBRV had minimal histopathological changes. To quantify apoptosis, brain sections from 4 mice infected with each virus were examined for apoptosis by using the TUNEL assay. Brain sections from sham-infected mice were also included. TUNEL-positive cells were counted in the cerebral cortex, hippocampus, thalamus, hypothalamus, brain stem and cerebellum from each mouse and the average number from four animals was obtained for each virus and analyzed statistically by one-way ANOVA and Student's t-Test. As summarized in Table 9, few apoptotic cells were observed in sham-infected animals. Apoptotic cells were observed in almost all the animals infected with each of the viruses. However, statistical analyses revealed that the number of apoptotic cells in mice infected with SHBRV was not significantly different from the number in sham-infected animals by either test. By one-way ANOVA, it was found that significantly more apoptotic cells (p<0.05) were observed in mice infected with B2C, RB2C, SN-10, and RSHBRV than in mice infected with N2C or RN2C (Table 9). However, Student t-test revealed that significantly more apoptotic cells (p<0.05) were observed in mice infected with B2C, RB2C, SN-10, RSHBRV, N2C and RN2C than in sham-infected mice. Furthermore, B2C and RB2C were statistically different (p<0.05) from all other groups. Overall, the recombinant viruses induced similar amount of apoptosis as the parental viruses from which the G was derived. Attenuated viruses (B2C, RB2C, and SN-10) induced more apoptosis than the pathogenic viruses (SHBRV, N2C, RN2C, and RSHBRV). Thus, induction of apoptosis inversely correlates with pathogenicity (FIG. 10). These data suggest that apoptosis is part of the host defense responses that normally play a protective role in rabies virus infection by restricting viral spread to the brain.

Higher Level of G Expression was Detected in Mice Infected with Attenuated than Pathogenic Viruses

The above studies indicate that the RV G is a major determinant for the induction of apoptosis. To determine if the induction of apoptosis was associated with the level of G expression as reported previously (Morimoto et al., 1999. J Virol 73: 510-518; Yan et al., 2001. J Neurovirol 7: 518-527; Faber et al., 2002. J Neurovirol 7: 518-527), viral antigens (G and the nucleoprotein [N]) were examined by immunohistochemical analysis. The level of G and N expression was scored and the results are summarized in Table 9. RV G was expressed abundantly in B2C- and RB2C-infected mice; a moderate level of G expression was detected in SN-10-, N2C-, and RN2C-infected animals, whereas the G expression was minimal in mice infected with SHBRV and RSHBRV. The level of G expression in the recombinant viruses is similar to that in the parental viruses from which the G is derived. On the other hand, the level of N expression was similar in animals infected with each of these viruses. N antigen was detected in almost all the neurons in the hippocampus, particularly in the CA3 region, although the antigen staining was less intense in mice infected with SHBRV than in mice infected with other laboratory adapted viruses (FIG. 9). Brain extracts from SHBRV- or B2C-infected mice were also subjected to PAGE and Western blot analysis, it was found that the level of G expression in SHBRV-infected mice was consistently three-fold lower than in B2C-infected mice while the level of N expression was similar in mice infected with either virus (Wang et al., 2005. J. Virol. 79:12554-12565; Example II). These results indicate that the level of G expression is associated with a particular RV strain and may correlate with the induction of apoptosis.

More Apoptotic Cells were Observed in Primary Neurons Infected with Attenuated than Pathogenic Viruses

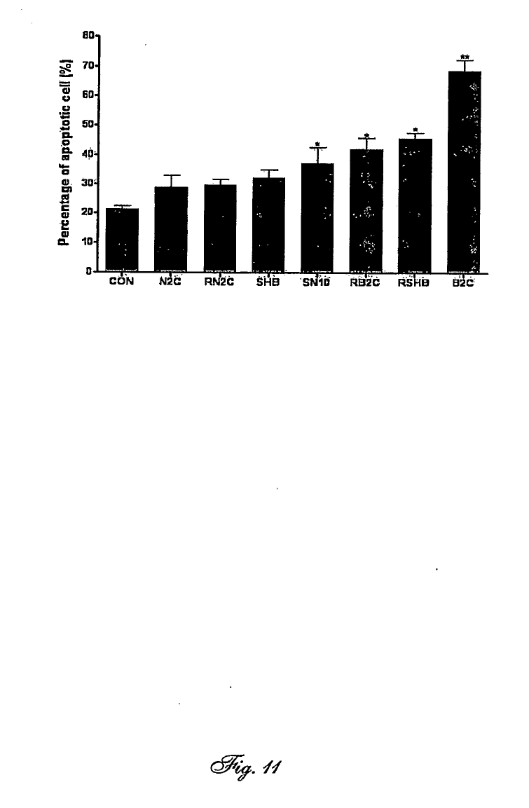

To determine if the induction of apoptosis in mouse brain correlates with in vitro studies, primary neuronal cultures were prepared as described (Adamec et al., 2001. Brain Res. Protocol 7:193-202; Li et al., 2005. J. Virol. 79:10063-10068). At day 7 after plating, cultured neurons were infected with each of the seven viruses at a multiplicity of infection (moi) of 0.1 ffu per cell and the cells were fixed with 4% paraformaldehyde and stained with either FITC-conjugated RV antibodies or with the TUNEL assay at day 5 post infection (p.i.) as described (Morimoto et al., 1999. J Virol. 73:510-518). By day 5 p.i., neurons showed almost 100% infection with each of the viruses. To assay apoptosis, TUNEL assay was performed in triplicate for each virus. The percentage of apoptotic neurons was determined in six 40× fields and presented in FIG. 11. Data were analyzed statistically by one-way ANOVA. Only neurons infected with CVS-B2C, RB2C, SN-10, or RSHBRV had significantly (p<0.05) more apoptosis than uninfected neurons whereas the number of TUNEL-positive neurons infected with SHBRV, CVS-N2C, RN2C were not significantly more than uninfected neurons. Student's t-test revealed significantly more apoptotic neurons in RN2C-infected cultures than sham-infected cultures and significantly more apoptotic cells (p<0.05) were detected in B2C-infected neurons than in neurons infected with any other virus (data not shown). Overall, the induction of apoptosis in primary neurons correlates with that in mice by these viruses.

Attenuated and wt RV Induced Different Clinical Signs after Intramuscular (IM) Infection

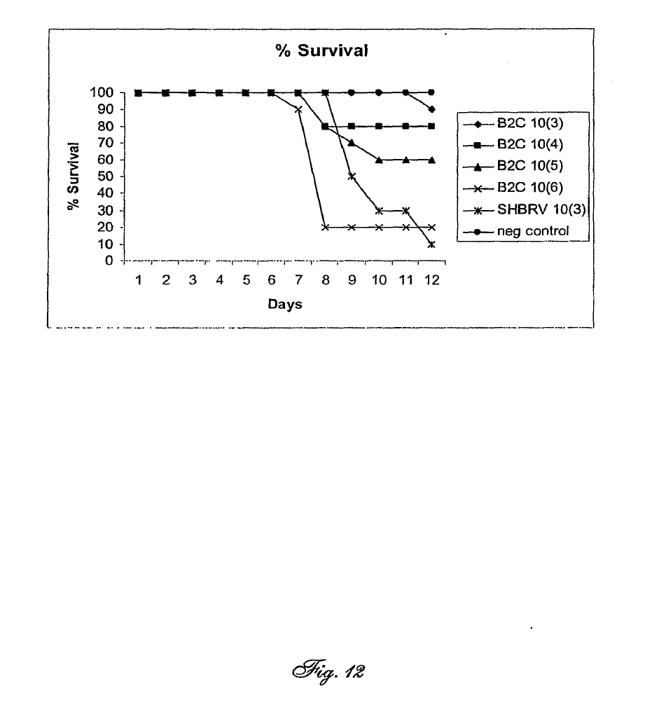

To investigate the mechanism by which induction of apoptosis attenuates RV pathogenicity, mice were infected with RV by IM and virus spread was monitored in the spinal cord and the brain. Two viruses, SHBRV and B2C, were selected. Initially the IMLD50 was determined for each virus by inoculation into both hind legs. FIG. 12 shows the survival curve of mice when infected with either the SHBRV at 10³ ffu or the CVS-B2C at 10³, 10⁴, 10⁵, 10⁶ ffu, respectively. At 10³ ffu, SHBRV killed 90% of the infected mice. However, only 10% of the mice infected with B2C succumbed to rabies at this dose. The mortality rate increased with increasing doses of B2C. At the dose of 10⁶ ffu, 80% of the mice infected with B2C succumbed to rabies. These data indicate that B2C is an attenuated virus and needs 3 logs more virus than SHBRV to kill a similar percentage of animals by IM.

Clinical signs were different in mice infected with these two viruses. In mice infected with B2C (10⁶ ffu), first sign of disease, ruffled fur, was observed on day 4 p.i. On day 5 p.i., mice had paresis and on day 6 p.i. flaccid paralysis of one or two hind limbs. Hunchback was observed as the disease progressed, followed by paralysis of the fore limbs. The mice then became prostrated with loss of muscle mass (wasting) and finally death beginning at day 7 p.i. (FIG. 12). In mice infected with SHBRV, on the other hand, no ruffled fur was observed. Paralysis was observed beginning on day 6 p.i. The paralysis in SHBRV-infected mice was different from that observed in B2C-infected mice. B2C-infected mice had flaccid paralysis while spastic paralysis was observed in SHBRV-infected mice. There was no voluntary joint movement, but involuntary spastic movement was common, and stimulation of the legs usually evoked a withdrawal reflex. As the disease progressed, the mice had hypersensitivity to noise and would jump vigorously or spin continuously until collapsing. In this situation, some would recover quickly while others would soon die. Mice infected with SHBRV began to die at day 8 p.i. (FIG. 12).

### Attenuated RV Induced Apoptosis in the Spinal Cord While wt RV did not

To monitor virus spread and the induction of apoptosis, mice were infected IM with SHBRV at 10³ ffu or B2C at 10³, 10⁴, 10⁵ and 10⁶ ffu. At 3, 5, 7, and 9 days p.i., brains and spinal cords were collected after transcardial perfusion. The tissues (4 mice in each group) were paraffin embedded and sectioned for detection of CD3-positiive T cells, apoptosis and viral antigen expression. No inflammatory cells were detected at days 1 and 3 p.i. Only a few CD3 positive T cells were observed in the spinal cord of mice infected with 10³ ffu of B2C at day 5 p.i. When mice were infected with 10⁴ ffu of B2C, more CD3-positive T cells were observed in the spinal cord, particularly at 5 and 7 days p.i. When mice were infected with 10⁵ ffu of B2C, more numbers of CD3-positive T cells were observed in the spinal cord, particularly at day 7 p.i. Mice infected with 10⁶ ffu of B2C showed most CD3-positive T cells during the investigation period. On the other hand, very few CD3 positive T cells were found in the spinal cord of mice infected with 10³ ffu of SHBRV. No CD3 positive cells were detected in sham-infected mice (Table 12).

TABLE 12
The number of CD3 positive cells observed in mice
infected through IM route with B2C or SHBRV.

| | The numbers of CD3 positive cells (mean ± SD) | | |
|---|---|---|---|
| | spinal cord | | |
| | Days p.i. | | |
| Virus/Dose | 5 | 7 | 9 |
| B2C 10³ | 0.6 ± 0.5 | 1.0 ± 0.7 | 0 |
| B2C 10⁴ | 3.0 ± 2.2 | 3.5 ± 1.3 | 1.3 ± 0.5 |
| B2C 10⁵ | 1.3 ± 0.5 | 12 ± 8.0 | 3.0 ± 2.1 |
| B2C 10⁶ | 8.0 ± 4.8 | 4.5 ± 2.9 | 16 ± 8.3 |
| SHB 10³ | 0.8 ± 0.8 | 1.5 ± 1.7 | 1.8 ± 2.2 |

| | |
|---|---|
| Control | 0 |

Since neither apoptosis nor viral antigen was observed in any mouse at day 3 p.i., no data are presented for this time point. The detection of apoptosis and antigen in the spinal cord and the brain are summarized in Tables 10 and 11, respectively. When mice were infected with 10³ of B2C, RV antigen was detected at day 5 p.i. in the spinal cord of 25% of the mice. However, RV antigen was detected in only a few neurons and their processes in the spinal cord and no RV antigen was detected in the brain.

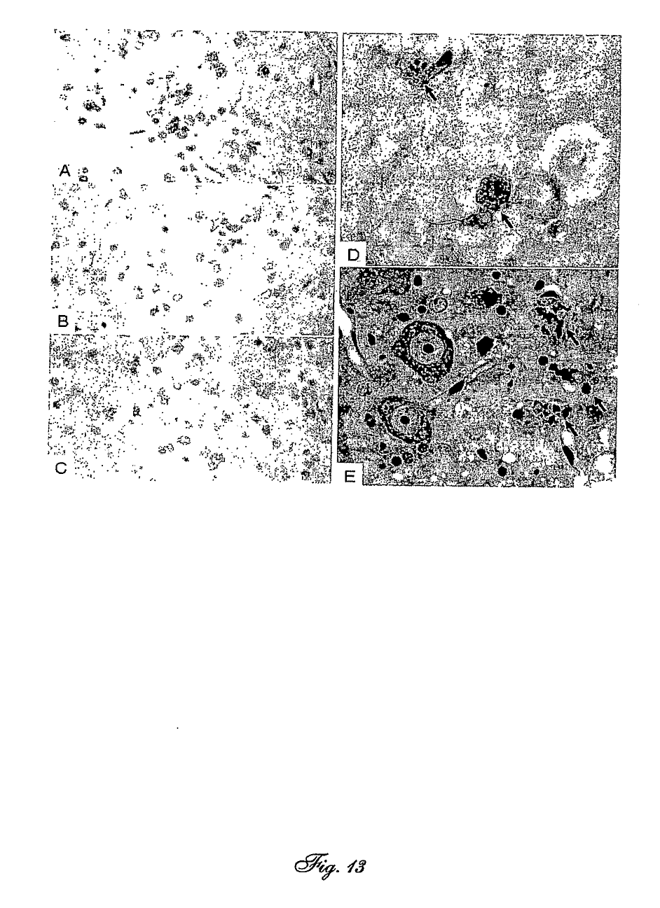

Furthermore, only a few TUNEL-positive cells were observed in the spinal cord of mice infected with 10³ ffu of B2C. When mice were infected with 10⁴ ffu of B2C, RV antigen was detected at day 5 p.i. in the spinal cord of 50% of the mice and the number of infected neurons increased when compared to mice infected with 10³ ffu of B2C. In addition, more apoptotic cells were observed in the spinal cord, particularly at day 5 p.i. By days 7 and 9 p.i., the number of apoptotic cells declined. In mice infected with 10⁵ ffu of B2C, RV antigen was observed in 75% of the animals in the spinal cord and 50% of the mice in the brain. In the spinal cord, about 50% of the neurons were infected. In the brain, RV antigen was detected in many neurons in the medulla, but in only a few neurons in the cerebral cortex and a few Purkinje cells in the cerebellum. Moderate numbers of apoptotic cells were observed in the spinal cord as well as in the brain, particularly at day 7 p.i. When mice were infected with 10⁶ of B2C, RV antigen was detected in all the infected animals in the spinal cord and most of the animals (75%) in the brain. Extensive RV antigen staining was detected in most of the neurons (80%) and their processes in the grey matter of the spinal cord at day 7 p.i. In the brain, RV antigen was also observed in most of the neurons in the medulla, 20% of the Purkinje cells in the cerebellum, and about 10% neurons in the cerebral cortex and thalamus/hypothalamus. Furthermore, apoptosis was detected in a moderate number of neurons at day 5 p.i. and extensive apoptosis at days 7 and 9 p.i. (Table 10, FIG. 13A).

When mice were infected with 10³ of SHBRV, on the other hand, RV antigen was detected in all animals in the spinal cord and most animals (75%) in the brain. In the spinal cord, RV antigen was more prominent in the neuropil than in perikarya. In the brain, RV antigen was detected in about 50% of the neurons in major brain regions, including the medulla, cerebellum, hippocampus, and cerebral cortex at days 7 and 9 p.i. Despite the fact that viral antigen was detected in almost all the animals in the spinal cord, little apoptosis was detected in the spinal cord or in the brain of mice infected with 10³ ffu of SHBRV (Table 10, FIG. 13B). No apoptosis was detected in sham-infected mice (FIG. 13C).

To confirm that infected neurons underwent apoptosis, double labeling was performed in the spinal cord for detection of viral antigen and apoptotic cells. As shown in FIG. 13D, double-labeled neurons were detected in mice infected with B2C. Furthermore, condensations of nuclear chromatin with neuronophagia were observed in the spinal cords of mice infected with B2C (FIG. 13E), but not in sham-infected mice or mice infected with SHBRV (data not shown).

### Discussion

Induction of apoptosis has been associated with RV G expression, particularly the level of G expression (Morimoto et al., 1999. J Virol 73: 510-518; Yan et al., 2001. J Neurovirol 7: 518-527; Faber et al., 2002. J Neurovirol 7: 518-527). Using a panel of recombinant RVs, we demonstrated that the induction of apoptosis is largely determined by the G. G gene also determines the level of G expression. Recombinant RVs expressed a similar level of the G and induced a similar level of apoptosis as the parental viruses from which the G was derived. For example, parental N2C and recombinant RN2C expressed a low level of G and only a few apoptotic cells were detected. On the other hand, parental B2C and recombinant RB2C expressed high levels of the G and induced extensive apoptosis. The level of G expression is not due to the rate of viral replication because the level of N expression is similar in animals infected with each of these viruses and N antigen is detected in almost all the neurons, particularly in the hippocampal region. However, recombinant RSHBRV induced significantly more apoptosis than the parental SHBRV, although the level of G expression is similarly low in mice infected with either virus. Although RV G alone from CVS has been shown to induce apoptosis in cell culture (Préhaud et al., 2003. J Virol. 77:10537-47) and recombinant RV expressing two copies of the G induced more apoptosis than RV expressing a single copy of the G (Faber et al., 2002. J Neurovirol 7: 518-527), recently it has been reported that RV matrix (M) protein alone is also capable of inducing apoptosis in neuroblastoma cells (Kassis et al., 2004. J Virol. 78:6543-55). In addition, the M proteins from other rhabdoviruses such as vesicular stomatitis virus (Kopecky et al., 2001. J Virol. 75:12169-81; Kopecky et al., 2003. J Virol. 77:5524-8) and infectious hematopoietic necrosis virus have also been reported to induce apoptosis. It is possible that the M in the SN-10 backbone contributed to the induction of apoptosis in the RSHBRV-infected mice. However, both recombinant RB2C and RN2C induced less apoptosis (albeit not significantly) than the parental B2C and N2C, respectively, whereas recombinant RSHBRV induced significantly more apoptosis than the parental SHBRV. These results may indicate that both G and M can independently induce apoptosis (Préhaud et al., 2003. J Virol. 77:10537-47; Kassis et al., 2004. J Virol. 78:6543-55), and also the interaction between the G and M may contribute to the induction of apoptosis in RV infections.

It has also been found that the ability of a particular RV strain to induce apoptosis inversely correlates with its pathogencity (Morimoto et al., 1999. J Virol 73: 510-518; Yan et al., 2001. J Neurovirol 7: 518-527; Pulmanausahakul et al., 2001. J Virol. 75:10800-7; Faber et al., 2002. J Neurovirol 7: 518-527). Overall the induction of apoptosis correlated inversely with pathogenicity among the seven parental and recombinant RVs tested in the present study (see FIG. 10). Attenuated viruses such as B2C induced extensive apoptosis while wt SHBRV induced little apoptosis in adult mice. Yet, a few viral particles of the SHBRV killed infected animals while 1000 to 10000 more viral particles were required by B2C to kill infected animals (see Table 9 and FIG. 10). Therefore our study confirms the previous hypothesis that induction of apoptosis is a host defense mechanism in RV infections (Morimoto et al., 1999. J Virol 73: 510-518; Yan et al., 2001. J Neurovirol 7: 518-527). However, the mechanisms by which induction of apoptosis protect RV-infected animals are not completely understood. It is possible that attenuated RV such as B2C, by inducing apoptosis, prevents virus spread within the CNS. To investigate such possibility, mice were infected with different doses of B2C by the IM route and the induction of apoptosis and virus spread were monitored in the CNS. It was found that lower doses of B2C resulted in mild RV infection limited to the spinal cord and infection of the spinal cord neurons induced apoptosis. This may explain why RV antigen was detected in only a few neurons in mice. Thus, we hypothesize that G-mediated induction of apoptosis limits the spread of attenuated rabies viruses in the CNS of mice. Although attenuated RV did not cause obvious neurological signs and death when given at lower doses, it is not known if induction of apoptosis in the spinal cord is associated with minor gait abnormalities or behavior changes.

In contrast, mice infected with low dose of SHBRV induced little apoptosis despite the fact that viral antigens were detected in most of the infected mice. Furthermore, a low dose of SHBRV resulted in spread to the brain and development of clinical rabies. Thus, our study suggests that induction of apoptosis is a host defense mechanism in RV infection that prevents virus spread from the spinal cord to the brain. Nevertheless, mice infected with high doses of attenuated RV developed neurological diseases and died of rabies. Thus, induction of apoptosis can have protective functions on one hand, but may also play a role in pathogenesis on the other, particularly in animals experimentally infected with high doses of attenuated RVs by the IC route (Jackson et al., 1997. J Virol 71:5603-5607; Theerasurakarn et al., 1998. J Neurovirol 4:407-414).

In addition to the induction of apoptosis, other pathological changes, particularly inflammatory reactions, were also detected in animals infected with attenuated RVs than pathogenic RVs. In our previous studies, significantly more CD3 positive cells were detected in mice infected with attenuated B2C and SN-10 than pathogenic SHBRV and N2C (Li et al., 2005. J. Virol. 79:10063-10068; Wang et al., 2005. J. Virol. 79:12554-12565; Example II). Infiltration of T cells has been reported to play a major role not only in blocking RV spreading in (Camelo et al. (2001). J Virol. 75:3427-34; Baloul et al. (2003) Biochimie. 85:777-88), but also in clearing RV from the CNS (Hooper et al. (1998). J Virol. 72:3711-9). Recently, we (Wang et al., 2005. J. Virol. 79:12554-12565; Example II) and others (Préhaud et al., 2005. J. Virol. 79:12893-12904) have shown that attenuated RVs induce strong innate immune responses such as up-regulation of IFN-α/β and inflammatory cytokines and chemokines. Furthermore it has been shown that virus uptake by neurons determines neuroinvasiveness as well (Faber et al. (2004). Proc. Natl. Acad. Sci. U S A. 101:16328-32) since it takes a shorter time for SHBRV to infect 50% of the cells in vitro than attenuated SN-10. Thus multiple factors may be involved in determining RV neuroinvasiveness and thus pathogenicity.

In the present study, severe pathological changes including apoptosis, inflammatory reaction, and gliosis were observed in mice infected with high doses of attenuated RVs, particularly B2C by either the IC or the IM route. On the other hand, only mild pathological changes were observed in mice infected with pathogenic RV such as SHBRV. The mild pathological changes observed in mice infected with pathogenic RVs resemble those observed in human patients who died of rabies (Murphy (1977) Arch Virol 54: 279-297). Furthermore, mice infected with SHBRV developed clinical signs different from those in mice infected with B2C. Mice infected with B2C developed ruffled fur, weight loss, and flaccid paralysis. The presence of neuronophagia, inflammation and gliosis, particularly in the spinal cord, correlates the clinical observations of a progressive, flaccid paralysis seen in mice infected with B2C and also reported in other virus infections, such as in humans infected with West Nile Virus (Kelley et al. (2003). Am J Clin Pathol. 119(5):749-53). On the other hand, mice infected with SHBRV developed hypersensitivity to environmental stimulus and died suddenly without any obvious signs. In addition, mice infected with SHBRV developed spastic paralysis, which has previously been reported in experimental wild-type rabies virus infection of mice (Jackson (1989) Neuropathol Appl Neurobiol 15: 459-475). Based on these findings, we propose that pathogenic and attenuated RVs employ different mechanisms to induce neurological diseases. In mice infected with attenuated RV, particularly at high doses, apoptosis and possibly inflammation play a major role in the development of neurological diseases. The induction of apoptosis and inflammation has been associated with the level of G expression as shown in this study as well as reported by others (Morimoto et al. (1999) J Virol 73: 510-518, Yan et al. (2001) J Neurovirol 7: 518-527, Préhaud et al. (2003). J Virol. 77:10537-47, Faber et al. (2002) J Neurovirol 7: 518-527; Faber et al. (2004) Proc. Natl. Acad. Sci. U S A. 101:16328-32, Wang et al., 2005. J. Virol. 79:12554-12565; Example II). How pathogenic RVs induce neurological disease without causing severe pathological changes remains to be determined.

TABLE 9
Virus titers, ICLD₅₀, IC pathogenic index, histopathological scores, the
number of apoptotic cells, and expression of viral antigens in the CNS of mice
infected with different RVs.

| | Virus | | Pathogenic | # of Apoptotic | Pathology | Viral N | Viral G |
|---|---|---|---|---|---|---|---|
| Viruses | Titers | ICLD50 | Index (IC) | Cells (±SD)* | Score¶ | antigen¶ | antigen¶ |
| SHBRV | 10^{5.23} | 10^{-4.5} | 0.19 | 5.25 ± 4.25 | + | +++ | + |
| N2C | 10^{5.11} | 10^{-4.7} | 0.38 | 14.75 ± 3.81 | ++ | +++ | ++ |
| RSHBRV | 10^{6.72} | 10^{-4.9} | 0.015 | 17.75 ± 4.75† | ++ | +++ | + |
| RN2C | 10^{8.87} | 10⁻⁶ | 0.0015 | 12.00 ± 3.44 | ++ | ++++ | ++ |
| B2C | 10^{7.94} | 10⁻⁵ | 0.0011 | 44.25 ± 16.29† | +++ | ++++ | ++++ |
| SN-10 | 10^{10.18} | 10⁻⁷ | 0.00066 | 20.50 ± 2.05† | ++ | ++++ | ++ |
| RB2C | 10^{10.71} | 10^{-5.8} | 0.000012 | 31.25 ± 23.75† | +++ | ++++ | +++ |
| Control | — | — | — | 2.75 ± 2.00 | − | − | − |

*TUNEL-positive cells were counted in the cortex, hippocampus, thalamus, hypothalamus, brain stem and cerebellum from each mouse and the average number from four animals was obtained for each virus and analyzed statistically by one-way ANOVA.
†significantly different from that of controls (P < 0.05)
¶Pathological lesions were scored according to the severity. +++ indicates extensive pathology with more than 50% of the neurons affected. ++ indicates observable pathology with 25-50% of the neurons affected. + indicates mild pathological changes with 10-25% neurons affected, while − indicates no pathological changes. The level of antigen expression is scored according to the immunostaining intensity and the number of neurons affected in the hippocampal region. ++++ indicates strong immunostaining with almost all the neurons affected. +++ indicates strong immunostaining with more than 50% neurons affected, or slightly weak staining with almost all the neurons affected. ++ indicates weak staining with about 25% neurons affected. + indicates weak staining with about 10% neurons affected. − indicates no detection of viral antigen.

TABLE 10
The number of apoptotic cells observed in mice infected through IM route
with B2C or SHBRV.

| | The numbers of apoptotic cells (mean ± SD) | | | | | |
|---|---|---|---|---|---|---|
| | spinal cord | | | brain | | |
| | Days p.i. | | | | | |
| Virus/Dose | 5 | 7 | 9 | 5 | 7 | 9 |
| B2C 10³ | 1.7 ± 1.5 | 1.7 ± 1.2 | 1.0 ± 1.7 | 1.2 ± 1.1 | 3.3 ± 1.0 | 1.3 ± 1.5 |
| B2C 10⁴ | 3.6 ± 2.2 | 1.5 ± 1.3 | 1.3 ± 0.5 | 1.0 ± 0.4 | 0.6 ± 0.5 | 1.0 ± 0.3 |
| B2C 10⁵ | 1.3 ± 0.5 | 20 ± 3.0 | 7.0 ± 2.1 | 2.3 ± 1.7 | 5.7 ± 1.1 | 2.0 ± 2.6 |
| B2C 10⁶ | 15 ± 4.8 | 33 ± 5.9 | 38 ± 3.3 | 1.3 ± 0.5 | 2.0 ± 0.4 | 3.5 ± 1.7 |
| SHB 10³ | 0.8 ± 0.8 | 1.9 ± 1.7 | 1.8 ± 2.2 | 1.3 ± 1.0 | 1.4 ± 0.7 | 1.6 ± 1.3 |
| Control | | 0.8 ± 0.5 | | | 1.3 ± 0.5 | |

Mice (four in each group) were infected with different doses of RV and spinal cords as well as brains were harvested at different time points after infection for TUNEL assay for detection of apoptosis. Apoptotic cells were counted in six fields in the spinal cord or six fields in the medulla, cerebral cortex and cerebellum. The average number of apoptotic cells and the standard deviation are shown.

TABLE 11
Detection of RV N antigen in different regions of the CNS (numbers of mice shown
positive staining/total animals tested).

| | Brain regions | | | | | | | | | | | | | | | | | |
|---|---|---|---|---|---|---|---|---|---|---|---|---|---|---|---|---|---|---|
| | | | | | | | | | | Thalamus/ | | | | | | | | |
| | | | | | | | | | | hypo- | | | | | | | | |
| | Spinal cord | | | medulla | | | cerebellum | | | thalamus | | | hippocampus | | | cortex | | |
| | Days p.i. | | | | | | | | | | | | | | | | | |
| Virus/Dose | 5 | 7 | 9 | 5 | 7 | 9 | 5 | 7 | 9 | 5 | 7 | 9 | 5 | 7 | 9 | 5 | 7 | 9 |
| B2C 10³ | 1/4 | 0/4 | 0/4 | 0/4 | 0/4 | 0/4 | 0/4 | 0/4 | 0/4 | 0/4 | 0/4 | 0/4 | 0/4 | 0/4 | 0/4 | 0/4 | 0/4 | 0/4 |
| B2C 10⁴ | 2/4 | 0/4 | 0/4 | 0/4 | 0/4 | 0/4 | 0/4 | 0/4 | 0/4 | 0/4 | 0/4 | 0/4 | 0/4 | 0/4 | 0/4 | 0/4 | 0/4 | 0/4 |
| B2C 10⁵ | 3/4 | 2/4 | 1/4 | 2/4 | 1/4 | 0/4 | 1/4 | 1/4 | 1/4 | 0/4 | 0/4 | 0/4 | 0/4 | 0/4 | 0/4 | 1/4 | 0/4 | 1/4 |
| B2C 10⁶ | 4/4 | 3/4 | 2/4 | 3/4 | 3/4 | 2/4 | 1/4 | 3/4 | 2/4 | 1/4 | 3/4 | 2/4 | 1/4 | 3/4 | 2/4 | 1/4 | 3/4 | 2/4 |
| SHB 10³ | 3/4 | 3/4 | 4/4 | 1/4 | 3/4 | 3/4 | 0/4 | 3/4 | 3/4 | 0/4 | 3/4 | 2/4 | 0/4 | 3/4 | 2/4 | 0/4 | 3/4 | 2/4 |

### Example V

### Recombinant Rabies Viruses that Express Interferon (IFN) and Chemokines

Since our studies found that activation of the innate immune responses, particularly IFN and chemokines, further attenuates RV virulence, we cloned IFN (α5 and β) and chemokine genes (MIP-1α, RANTES, and IP-10) into two different viral genomes.

### Construction of Recombinant HEP Rabies Virus Expressing IFN and Chemokines

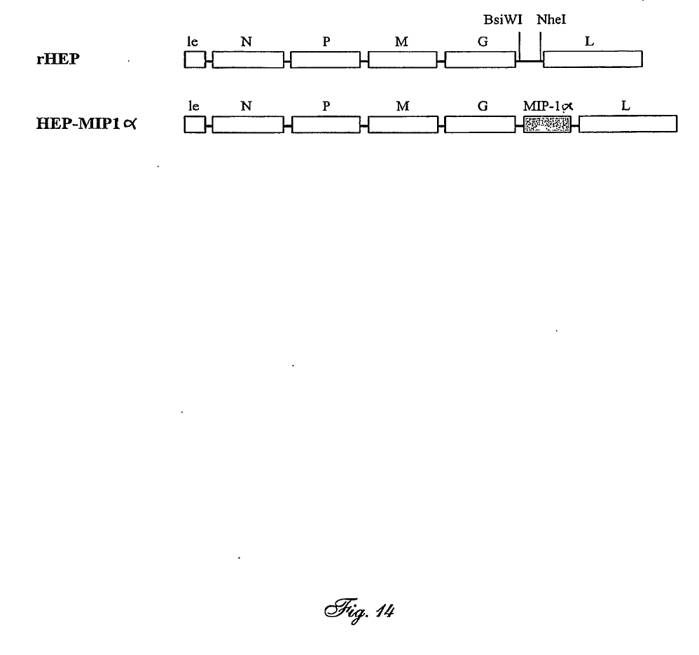

Mouse IFN-β and chemokines (MIP-1α, RANTES, and IP-10) were amplified by RT-PCR (reverse transcription polymerase chain reaction) from mouse brain and cloned into pHEP-3.0 (the infectious clone derived from rabies virus strain flurry HEP). HEP is one of the most attenuated rabies virus and has been used as a vaccine strain for rabies virus vaccine in parts of the world. Each of the IFN or chemokine genes was inserted between the G and L genes in the rabies virus genome. An example for HEP-MIP1α is shown in FIG. 14.

### Recovery of Recombinant Rabies Viruses Expressing IFN and Chemokines

Recombinant viruses were recovered in BSR cells which were transfected with each of the recombinant plasmids (pHEP-MIP-1≢, pHEP-IFN-β, pHEP-RANTES, and pHEP-IP 10) together with plasmids expressing N, P, and L. Recovery of these recombinant rabies viruses was confirmed by immunofluorescent antibody assay with anti-rabies virus N antibodies. Confirmation of these recombinant rabies viruses was carried out by obtaining the recombinant viral genomic RNA and sequencing the inserted genes. All these recombinant viruses were successfully recovered.

Comparison of Growth Characteristics between Parental Virus and Recombinant Rabies Viruses

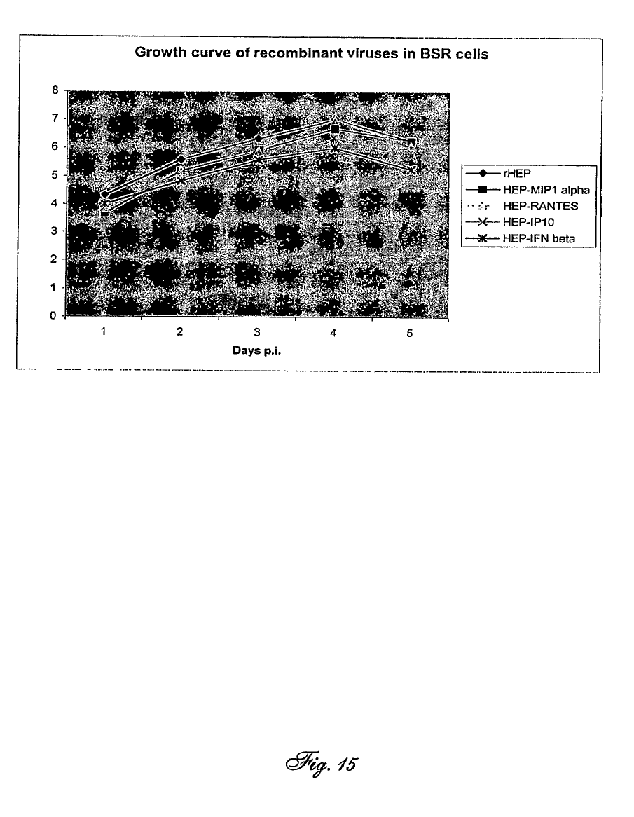

To compare the growth characteristics between parental and recombinant rabies viruses, BSR cells were infected with each of the viruses at the same dose. At 1, 2, 3, 4 and 5 days after infection, virus titers were determined from the supernatants. It was found that each of these recombinant rabies viruses has similar growth curves (FIG. 15) as the parental virus, indicating that these recombinant rabies viruses have similar growth characteristics as the parental virus. In another word, expression of an extra gene in the rabies virus genome did not affect virus growth and yield in cell culture, very important for vaccine development.

### Confirmation of Recombinant Viruses in Producing the Intended Chemokines in Infected Cells

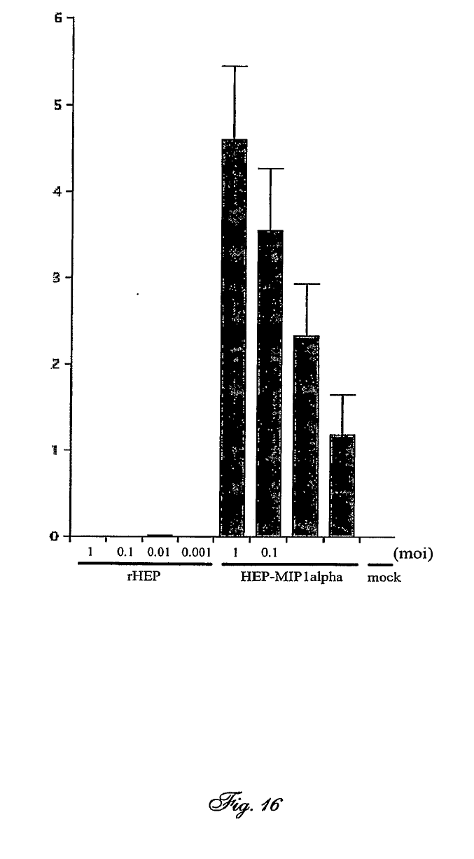

To confirm that the recombinant rabies viruses expressed the intended products, NA cells were infected with recombinant virus HEP-MIP1α. At 24 hour incubation, the culture supernatants were harvested and assayed for the expression of MIP-1α by MIP-1α ELISA kit. As shown in FIG. 16, MIP-1α is expressed only in cells infected with the recombinant rabies virus HEP-MIP1α, but not in mock-infected cells or cells infected with parental virus (rHEP). This indicates that recombinant rabies virus is capable of producing the intended products. For other recombinant rabies viruses, confirmation is still under way.

### Construction of Recombinant L16-G Rabies Virus Expressing IFN and Chemokines

Two IFN (α5 and β) and two chemokine genes (MIP-1α and IP-10) were also cloned into L16-G genome because L16-G is essentially SAG2 and is the most attenuated RV. These genes (α5 and β, MIP-1α and IP-10) were selected because of their high level of expression in mice infected with attenuated RV, but not in virulent RV-infected mice. We used the construct pGEM-GHL with a unique Hpal site between G and L and cloned the IFN or chemokine genes at the HpaI site with proper gene initiation and termination, as well as intergenic sites. After confirmation, the resulting plasmid was digested with XhoI and the fragment was cloned back to pL16-GΔXhoI. So far, we have obtained infectious clones L16-G/IFNA5, L16-G/IFNB, L16-G/MIP1A, and L16-G/IP10. In addition, L16-G/MIP1A recombinant virus has been selected and confirmed to have the correct insertion and in vitro characterization has shown that this recombinant virus grow to similar titers as the parental virus L16-G in BSR cells.

### Example VI

### Development of Avirulent Rabies Virus that Expresses Interferon (IFN) and Chemokines for Use as a Vaccine

To increase the immunogenicity of live avirulent RV vaccines, interferons (e.g., IFN-β) or select chemokine genes can be cloned into an avirulent RV genome, as shown in Example V. These recombinant viruses can be characterized in vitro for growth characteristics and stability. These constructs can also be tested in vivo for expression of the relevant IFN-β or chemokines, activation of the innate and enhancement of the adaptive immune responses, as well as for protection against challenge infection with virulent RV.

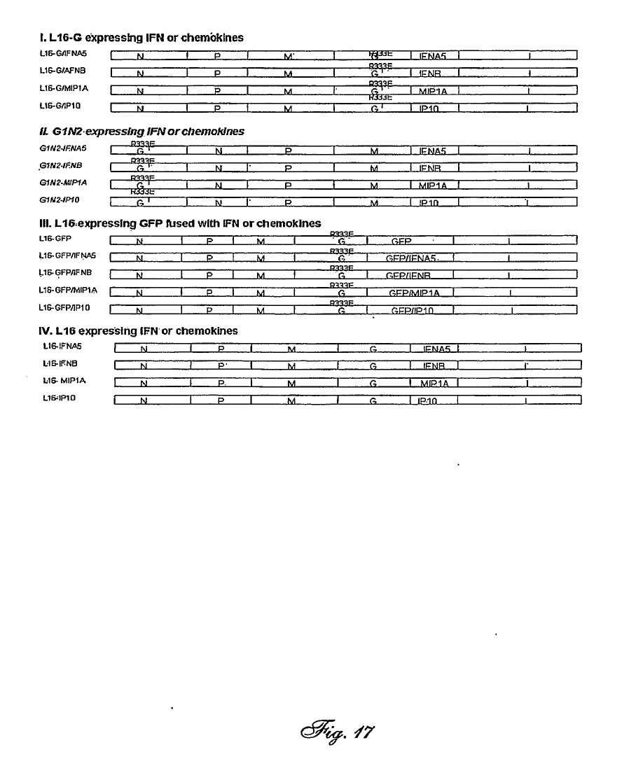

We chose L16-G as the backbone for cloning the IFN and chemokine genes (Example V) because L16-G is essentially SAG2 (group I virus) and thus does not induce any disease in adult animals by any route of inoculation. The virus is capable of invading the first order of neurons (motor or sensory), but invasion of second order of neurons is inhibited. Virus replication is limited to local injection site and does not spread to neighboring regions even after direct sterotaxic injection into the hippocampus. Furthermore, we will construct recombinant G1N2 viruses expressing IFN and chemokines as group II viruses. These viruses should increase the adaptive immune responses because the G is relocated to the 1^{st} position. Since R is mutated to E on the G, we do not expect any virulence from the groups I and II constructs. Since IFN (about 200 residues) and chemokines (70-90 residues) are small peptides, fusion proteins can be constructed in which each of the IFN and chemokine is fused with GFP (Group III viruses, FIG. 17). Recombinant RV expressing these fusion proteins can deliver IFN or chemokine and also provide a convenient way for virus detection in vivo.

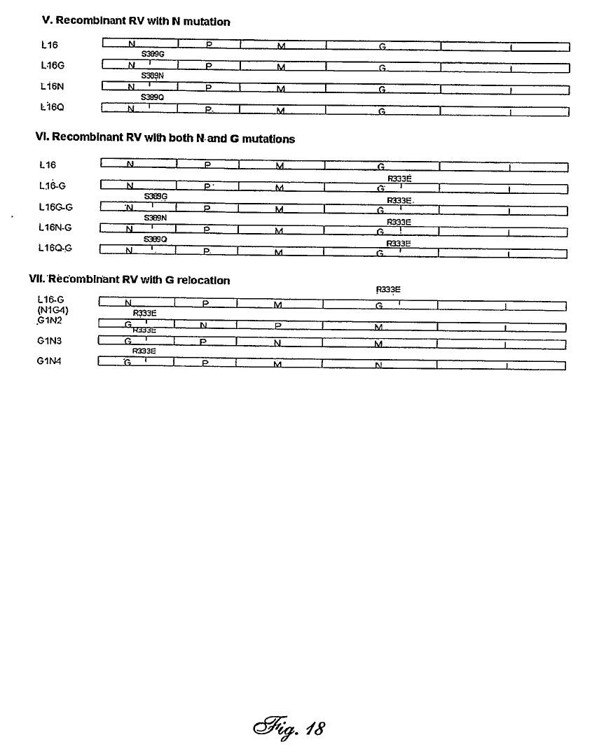

Group IV viruses are based on the L16, which is the parental virus from which SAG2 is derived (Flamand et al., 1993. Trends. Microbiol. 1:317-320). L16 virus has residual virulence and can induce neurological disease by IC challenge in adult mice. We construct this group of viruses for the purpose of comparison. In addition, we have already constructed recombinant infectious clones with mutation of the N (pL16G, pL16N, and pL16Q) (group V viruses), with mutation on both the N and G (pL16-G, pL16G-G, pL16N-G, and pL16Q-G) (group VI viruses), and with G relocated to the 1^{st} position (pGIN2). It has been reported by Flanagan et al. (Flanagan et al., 2000. J. Virol. 74:7895-7902; Flanagan et al., 2001. J Virol. 75:6107-14; Flanagan et al., 2003. J. Virol. 77:5740-5748) that VSV with G1N2 relocation still induced disease and thus we will construct G1N3 and G1N4 (group VII viruses, FIG. 18). Inclusion of these groups of viruses will permit further attenuation.

Once the constructs are made, recombinant RVs will be selected as described (Wu et al., J. Virol. 2002, 76:4153-4161). The virus will be propagated and PCR and sequence analysis will be performed to make sure that these viruses have the correct sequence. Each of these viruses will be tested in vitro for their rate of replication (Northern blotting), virus production (growth curve), and stability (20 in vitro passages monitored by RT-PCR and sequence analysis every 5^{th} passage) when compared with the parental virus L16 as described. Furthermore, BSR cells will be infected with different concentration (0.1, 1, and 10 ffu/cell) of these viruses and harvested at different time points (24, 48, and 72 hr p.i.). Supernatants will be used for measurement of the production of IFN or chemokines with commercial ELISA tests. To make sure that the expressed IFN or chemokines are biologically active, IFN activity will be measured by their ability to inhibit infection of Newcastle disease virus (NDV) expressing GFP in CEF cells (Park et al., 2003. J Virol. 77:1501-11), (kindly supplied by Dr. Peter Palese, Mount Sinai School of Medicine, NY). NDV-GFP is very sensitive to IFN treatment. Chemokine activity will be detected by their ability to recruit leucocytes using a modified Boyden chamber system as described (Tripp et al., 2001. Nat Immunol 2:732-8). The cell pellets will be used for protein extraction and evaluation of relevant IFN or chemokine transcripts by RT-PCR or Northern blot hybridization.

Most of the infectious clones have been constructed, and many viruses have already been selected. Since all the recombinant RV expressing IFN and chemokines were constructed without N mutation, we expect that the recombinant RV expressing IFN and chemokines should grow well in cells as the parental virus. It is expected that recombinant RV will express the intended IFN or chemokine with biological activities in a dose-dependent manner.

Determination of the virulence and pathogenicity of recombinant RV that expresses IFN-β or chemokines. For determination of virulence and pathogenicity, both adult and suckling mice (group of 10) will be inoculated with each virus at 10², 10³, 10⁴, or 10⁵ ffu by the IC route. Animals will be observed daily for 4 wk for development of rabies. Body weight will be monitored daily for adult mice. Mobility and mortality will be scored as follows: 0=normal mice, 1=ruffled fur, 2=loss of agility, 3=one paralyzed hind leg, 4=two paralyzed hind legs, 5=total paralysis (defined as the total loss of mobility), and 6=death. Any sick mouse or mouse dying of rabies will be euthanized and brains used for RNA extraction, which will be used for PCR and sequencing of the mutated site to determine if reversion occurs. It is our goal to develop avirulent rabies virus vaccines, which means that inoculation into adult and suckling mice will not induce rabies even by the IC route of infection. All the data will be analyzed statistically by student T test, X² test, or one way ANOVA.

Since our recombinant viruses express IFN and chemokines, these viruses may induce pathology, particularly histological lesions, although they may not induce obvious disease. Histopathology may eventually lead to chronic diseases. It has been reported that chemokine treatment can limit MHV replication, but may also induce demyelination. Thus we will investigate the pathology induced by our recombinant RVs. Mice (4 in each group) infected with different doses of each virus by either IC or by IM will be sacrificed at 1, 2, 3, and 4 wk after infection, which allows us to detect short- and long-term effects of the expressed chemokines. After perfusion, brains as well as spinal cords will be removed for histological analyses. Pathological lesions will be scored according to the severity. 3=extensive pathology with more than 50% of the neurons affected. 2=observable pathology with 25-50% of the neurons affected. 1=mild pathological changes with 10-25% neurons affected, 0=no pathological changes. Particular attention will be paid to apoptosis, necrosis, gliosis, and demyelination (also see D2). If any of these lesions is observed, quantitation of affected cells will be performed as described (Li et al., 2005, J. Virol. 79:10063-10068; Sarmento et al., 2005, 1. Neurovirol. 11:571-81) and will be compared to sham-infected mice by statistical analysis.

Among these 7 groups of viruses, groups I and II viruses are our intended vaccine candidates. Group I viruses are constructed on the backbone of SAG2 and thus should be avirulent even for suckling mice. If this occurs, it will indicate that expression of IFN or chemokine can further attenuate RV. Group II viruses should not induce disease in adult mice and they may not induce diseases in suckling mice, either. Since G1N2 VSV construct caused death in infected animals, but G1N2 VSV is less virulent than the parental virus (Flanagan et al., 2000. J. Virol. 74:7895-7902). G1N2 RV may thus be less virulent than L16-G (SAG2) virus. Group III viruses will be used in vivo studies to assist virus detection while group IV viruses will be used for comparison with group I viruses. Group IV viruses may still induce diseases in adult mice by the IC route, but will be attenuated when compared to L16. If group IV viruses do not induce any diseases, it will indicate that IFN and chemokine can reduce dramatically RV virulence. For group V-VI viruses, we should demonstrate whether N mutation alone at the phosphorylation site is enough or mutation on both the N and G are required to make RV avirulent. Group VII viruses should not induce disease in adult or suckling mice since all these viruses bear mutation on G333. If this occurs, it will indicate that relocation of the G and N on RV genome can further attenuate RV.

Determination of the immunogenicity of recombinant RV that expresses IFN-β or chemokines. For determination of immunogenicity, adult mice (group of 10) will be inoculated with each virus at 10², 10³, 10⁴, and 10⁵ ffu by the IM route (usual route for RV vaccination). It has been known for a long time that the protective mechanism against rabies is virus neutralizing antibodies (VNA) (Hooper et al., 1998. J Virol. 72:3711-9). To investigate the immunogenicity of the recombinant RV, we propose to determine how long the infected virus will be replicating in the CNS, how quick and how strong the VNA response will be. To determine how long the recombinant RVs replicate in the CNS, infected mice will be sacrificed at day 0, 1, 3, 5, 7, 9, and 11 days after infection, perfused brains and spinal cord will be subjected to immunohistochemistry for detection of viral antigens as described (Sarmento et al., 2005, J. Neurovirol. 11:571-81; (Yan et al., 2002. J Neurovirol. 8:345-52). Alternatively fresh frozen brains and spinal cords will be obtained and in situ hybridization will be performed to detect viral RNA as described (Fu et al., 1993. J. Virol. 67:6674-6681). To determine the speed and strength of the immune responses induced by the recombinant RVs, blood samples will be obtained before and 1, 2, 3, and 4 wk after inoculation for measurement of VNA as described (Tims et al., 2000. Vaccine. 18:2804-7). Ultimately, immunogenicity is determined by the rate of protection against lethal challenge. Thus, inoculated mice will be challenged with virulent CVS-24 one week after the last blood collection. All the data obtained from infection with different viruses will be analyzed statistically.

It should be noted that RV vaccines are usually given to victims after RV exposure in conjunction with anti-RV immunoglobulin. We expect that recombinant RV expressing chemokines or IFN-J3 can induce innate immune responses which block virulent virus spread within the CNS when mice are challenged with lethal RV immediately before or at time points after vaccination. Recombinant RV expressing chemokines or IFN-13 could have the potential to eliminate the need for anti-RV immunoglobulin, which is in short supply.

### Summary

Our studies show that mutation of the N at the phosphorylation site results in reduction of viral replication. Reduction of viral replication in vitro can be translated into RV attenuation in vivo, suggesting that mutation of the N can lead to reduction of virulence. Previous studies have demonstrated that mutation of the G can reduce neuroinvasiveness (Lafay et al., 1994, Vaccine 12:317-320). These results demonstrate the feasibility to develop avirulent RV vaccines by constructing mutation on both the N and G. Our studies also indicate that mutation of a single nucleotide of one amino acid is easily reversible, which led us to construct mutations of at least two nucleotides on one codon. Our studies also showed that it is CK-II that phosphorylates RV N in the infected cells. Furthermore, our studies indicate that N phosphorylation occurs after RNA encapsidation and we thus hypothesize that N phosphorylation facilitates subsequent round of viral replication (Liu et al., 2004, J Gen Virol. 85:3725-34). By mutation of the N at the phosphorylation site, we reduced the rate of viral replication and thus attenuated the virus.

Significantly, we found that attenuated RV activates, while virulent RV evades, the host innate immune responses. As detected by the microarray technology and real time PCR, almost all the genes involved in the activation of the IFN-β pathway and many of the inflammatory chemokines were up-regulated in animals infected with attenuated L16 and B2C by either the IC or IM routes. However, many of these genes are not up-regulated in animals infected with virulent SHBRV. For those genes involved in the IFN-β pathway that are up-regulated in SHBRV-infected animals, the magnitude of increase is at least 2 to 30-fold lower than that in B2C- or L16-infected mice.

Up-regulation of IFN can have direct anti-viral activities. Previously, reports have shown that IFN treatment has various degree of protection against RV infection in mice, hamsters, rabbits or monkeys (Harmon et al., 1974. Antimicrob Agents Chemother. 6:507-11; Hilfenhaus et al., 1975. Infect Immun.11:1156-8). Hooper et al. (1998, J Virol. 72:3711-9) reported that higher RV titers were detected in IFN-β receptor knockout (IFNAR^{-/-}) mice than immunologically intact mice. We also show that virulent RV is more sensitive to IFN treatment than attenuated RV. Up-regulation of inflammatory chemokines can recruit neutrophils, monocytes, and lymphocytes (Alcami, 2003, Nat Rev Immunol. 3:36-50; Rossi et al., 2000. Annu Rev Immunol. 18:217-42). Indeed, more CD3-positive T cells were detected in the brain or the spinal cord of mice infected with attenuated than with virulent RV (Example II; Li et al., 2005, J. Virol. 79:10063-10068; Wang et al., 2005. J. Virol. 79:12554-12565). Inflammatory reaction and infiltration of T cells have been reported to play a major role in RV clearance from the CNS (Hooper et al., 1998. J Virol. 72:3711-9). Furthermore, we showed that inflammatory reaction in the spinal cord prevented RV from spreading to the brain. All these data indicate that innate immune responses results in RV attenuation. Thus we expect expression of IFN and chemokines in RV genome to induce innate immune responses and at the same time to enhance the adaptive immune responses. Such recombinant RV (some have been constructed and selected as described above) can be developed as live and avirulent vaccines.

The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. An attenuated rabies virus comprising a polynucleotide operably encoding at least one mammalian immune factor.

2. The attenuated rabies virus of claim 1 wherein the polynucleotide operably encodes a plurality of mammalian immune factors.

3. The attenuated rabies virus of claim 1 wherein the immune factor is selected from the group consisting of an interferon, a cytokine and a chemokine.

4. The attenuated rabies virus of claim 3 wherein the interferon comprises an IFN-α, IFN-β or IFNγ interferon.

5. The attenuated rabies virus of claim 3 wherein the chemokine is selected from the group consisting of MIP-1α, MIP-1β, MCP, RANTES and IP-10.

6. The attenuated rabies virus of claim 3 wherein the polynucleotide further operably encodes an immune factor comprising either or both of a Toll-like receptor (TLR) or a TLR adaptor molecule.

7. A pharmaceutical composition comprising:
the attenuated rabies virus of claim 1; and
a pharmaceutically acceptable carrier.

8. The pharmaceutical composition of claim 7 which, following administration to a mammalian subject, induces an immune response in the subject that blocks or inhibits the spread of a pathogenic rabies virus within the central nervous system of the subject.

9. The pharmaceutical composition of claim 7 formulated for use as a live vaccine for the vaccination of a mammalian subject against rabies.

10. The pharmaceutical composition of claim 9 wherein the amount of attenuated rabies virus is effective to induce neutralizing antibodies in a subject.

11. The pharmaceutical composition of claim 9 wherein the amount of attenuated rabies virus is effective to protect the subject against experimental or natural challenge with a pathogenic rabies virus.

12. A method for vaccinating a mammalian subject comprising administering to the mammalian subject the pharmaceutical composition of claim 7.

13. The method of claim 12 wherein the pharmaceutical composition is administered prior to exposure to a pathogenic rabies virus.

14. The method of claim 12 wherein the pharmaceutical composition is administered after to exposure to a pathogenic rabies virus.

15. The method of claim 12 wherein the subject is a human.

16. The method of claim 12 wherein the subject is a domestic or wild animal.

17. A pharmaceutical composition comprising:
an attenuated rabies virus; and
a pharmaceutically acceptable carrier.

18. The pharmaceutical composition of claim 17 which, following administration to a mammalian subject, induces an immune response in the subject that blocks or inhibits the spread of a pathogenic rabies virus within the central nervous system of the subject.

19. The pharmaceutical composition of claim 17 formulated for use as a live vaccine for the vaccination of a mammalian subject against rabies.

20. The pharmaceutical composition of claim 19 wherein the amount of attenuated rabies virus is effective to induce neutralizing antibodies in a subject.

21. The pharmaceutical composition of claim 19 wherein the amount of attenuated rabies virus is effective to protect the subject against experimental or natural challenge with a pathogenic rabies virus.

22. A method for vaccinating a mammalian subject comprising administering to the mammalian subject the pharmaceutical composition of claim 17.

23. The method of claim 22 wherein the pharmaceutical composition is administered prior to exposure to a pathogenic rabies virus.

24. The method of claim 22 wherein the pharmaceutical composition is administered after to exposure to a pathogenic rabies virus.

25. The method of claim 22 wherein the subject is a human.

26. The method of claim 22 wherein the subject is a domestic or wild animal.
